(19) United States
(12) Patent Application Publication
(10) Pub. No.: US 2023/0255872 A1
(43) Pub. Date: Aug. 17, 2023

(54) ORAL CARE COMPOSITIONS AND METHODS

(71) Applicant: DENTHERAPY LTD, Aberdeen (GB)

(72) Inventor: Richard Willson, Plymouth (GB)

(73) Assignee: DENTHERAPY LTD, Aberdeen (GB)

(21) Appl. No.: 18/015,926

(22) PCT Filed: Jul. 14, 2021
(86) PCT No.: PCT/GB2021/051807

§ 371 Date: Jan. 12, 2023

(30) Foreign Application Priority Data
Jul. 16, 2020 (GB) 2010987.2

(51) Int. Cl. A61K 8/64; A61Q 11/00

(52) U.S. Cl. CPC A61K 8/64; A61Q 11/00; A61K 2800/28; A61K 2800/92; A61K 2800/48

(57) ABSTRACT

The present invention relates to compositions comprising a phosphopeptide for use in preventing or reducing staining of an oral surface, for prolonging the effect of a whitening agent on an oral surface and for use in remineralisation or prevention of demineralisation of an oral surface, wherein the composition does not comprise additional phosphate or an additional source of phosphate. The invention also extends to various compositions comprising a phosphopeptide that are substantially fluoride free.

The present invention relates to compositions for use in oral care, to methods of their production and various methods of their use in. In particular, the invention relates to compositions comprising phosphoproteins and their use to remineralise or prevent demineralisation of dental structures and/or to their use to prevent or reduce staining of oral surfaces.

### Background of the Invention

The following discussion is provided to aid the reader in understanding the disclosure and does not constitute any admission as to the contents or relevance of the prior art.

Dental enamel and dentine are subject to processes of demineralisation, characterised by loss of calcium and phosphate from the porous tooth surface, and remineralisation whereby the actions of saliva restore the hydroxyapatite of the tooth enamel. When the rate of demineralisation is greater than remineralisation, in the early phase, demineralisation causes the softening of the enamel making it susceptible to enamel loss. Progression from early phase demineralisation can result in cariogenic lesion or dental erosion. Enamel erosion and dental caries is typically caused by the demineralisation of dental enamel, typically by organic acids produced from fermentation of dietary sugar by dental plaque bacteria or from acid foods or drink, or from intrinsic acids during gastric reflux or bulimia nervosa.

Tooth enamel consists mostly of calcium hydroxyphosphate, Ca₅(PO₄)₃OH (also known as hydroxyapatite). Hydroxyapatite is a hard, insoluble compound, but acid in the mouth (e.g. from dental plaque bacteria) breaks down the apatite. The chemical reaction is as follows: Ca₁₀(PO₄)₆(OH)₂+8H+=10Ca²⁺+6HPO₄²⁻+2H₂O (See, “On the Dissolution of Hydroxyapatite in Acid Solutions”: J.Dent.Res. 1988, 76, 1056).

In an acidic environment (i.e. at a pH below the pka of hydroxyapatite) the enamel will begin the process of dissolution where calcium, phosphate and hydroxyl groups will become ionised and solvated by the surrounding solvent. The process is an equilibrium between the calcium, phosphate and hydroxide molecules in a solid crystal lattice and the corresponding ions in solution. The position of the equilibrium is dictated by: the pKa of the dissolving apatite, the pH of the solution and by the concentration of the ions in solution.

The addition of fluoride has the advantage of inducing a chemical modification of enamel from calcium hydroxyl apatite to calcium fluorapatite and so change the position of the dissolution equilibrium to favour the solid form of apatite. In addition the availability of fluoride ions act as a catalyst to promote the remineralisation of eroded enamel. Fluorapatite resists attacks by acids better than hydroxyapatite itself, so the tooth enamel resists decay better than enamel containing no fluoride.

Thus, it is known that remineralisation can be enhanced by inclusion of a source of fluoride ions in dental care formulae. It is also known that delivery of bioavailable calcium and phosphate ions to the enamel surface enhances remineralisation by changing the position of the equilibrium to favour the apatite crystalline form. However, these ions are prone to forming insoluble salts upon mixing in aqueous solution. Therefore, a simple solution containing calcium, phosphate and fluoride will react with itself to form an insoluble calcium fluoride solid that is not biologically active.

WO1998/040406 and WO2006/050013 teach that soluble complexes of amorphous calcium phosphate (ACP) or amorphous calcium phosphate fluoride (ACPF), stabilised by phosphopeptides containing the amino acid sequence Ser(P)-Ser(P)-Ser(P)-Glu-Glu, may be formed by admixing of solutions of calcium, phosphate and fluoride ions with solutions of phosphopeptides at pH levels above or below neutral respectively. Such preparations are suitable for inclusion in medicaments for the prevention or treatment of dental cariogenic conditions by delivering bioavailable ACP or ACPF to the enamel surface. WO2006/135982 teaches that phosphopeptide stabilised ACP or ACPF can be ‘superloaded’ with calcium and phosphate ions by mixing purified stabilised complexes with a suitable source of calcium and phosphate ions for the production of medicaments intended to prevent or treat dental caries. Oral surfaces are also subject to discolouration for a number of reasons. Staining is a common form of discolouration caused by certain foods and drinks. Foods, such as vegetables that are rich with carotenoids or xanthonoids, and coloured liquids, such as sports drinks, cola, coffee, tea and red wine, smoking, betel nuts and chewing tobacco can stain teeth. Staining commonly affects dental enamel, but can also affect other oral surfaces, e.g. dental restorations (e.g., composite fillings, veneers, crowns) or prosthetics, or orthodontic aligners and appliances.

US20060292090 describes tooth remineralizing and whitening devices and compositions where amorphous calcium phosphate is used to remineralise teeth and conventional teeth whiteners such as peroxides are used to whiten teeth. WO2007/111616 describes oral care compositions comprising linear condensed polyphosphate polymers and phytate in combination with a tooth bleaching agent such as peroxide to provide enhanced whitening.

There is a need for additional compositions and methods to address the individual issues of (i) demineralisation and (ii) staining, which may well occur in combination.

### SUMMARY OF THE INVENTION

In a first aspect there is provided a composition comprising a phosphopeptide for use in preventing or reducing staining of an oral surface.
In some embodiments, the composition does not comprise fluoride.

In a second aspect there is provided a method of preventing staining of an oral surface comprising contacting said surface with a composition comprising a phosphopeptide.
In one embodiment, the composition may be used in combination with a whitening agent.
In one embodiment, the oral surface is contacted with a whitening agent prior to contact with the composition.
In one embodiment, the composition does not comprise fluoride.

In a third aspect there is provided a composition comprising a phosphopeptide for use in prolonging the effect of a whitening agent on an oral surface.
In some embodiments, the composition does not comprise fluoride.

a fourth aspect, there is provided a method of prolonging the effect of a whitening agent on an oral surface, comprising contacting said surface with a composition comprising a phosphopeptide.
In one embodiment, the oral surface is contacted with a whitening agent prior to contacting with the composition comprising a phosphopeptide.
In some embodiments, the composition does not comprise phosphate or a source of phosphate.
Suitably, in some embodiments, the composition does not comprise fluoride.

In a fifth aspect there is provided an oral care composition comprising a phosphopeptide, and optionally monofluorophosphate, wherein the composition does not comprise additional phosphate or an additional source of phosphate.
In some embodiments, the oral care composition may act to remineralise an oral surface or prevent demineralisation of an oral surface. Suitably therefore, the oral care composition may be for use in remineralizing and/or preventing demineralisation of an oral surface. Suitably in such an embodiment, the oral care composition comprises calcium.
Suitably, in one embodiment, the oral care composition does not comprise fluoride.

In a sixth aspect there is provided a composition comprising a phosphopeptide and optionally monofluorophosphate, for use in remineralisation or prevention of demineralisation of an oral surface, wherein the composition does not comprise additional phosphate or an additional source of phosphate.
In some embodiments, the composition does not comprise fluoride. Suitably therefore a preferred embodiment of the invention is a composition comprising a phosphopeptide for use in remineralisation or prevention of demineralisation of an oral surface, wherein the composition does not comprise additional phosphate or an additional source of phosphate, and wherein the composition does not comprise fluoride.

In a seventh aspect there is provided a method of remineralisation or prevention of demineralisation of an oral surface, the method comprising contacting said oral surface with a composition comprising a phosphopeptide, and optionally monofluorophosphate, wherein the composition does not comprise additional phosphate or an additional source of phosphate.
In some embodiments, the composition does not comprise fluoride. Suitably therefore a preferred embodiment of the invention is a method of remineralisation or prevention of demineralisation of an oral surface, the method comprising contacting said oral surface with a composition comprising a phosphopeptide, wherein the composition does not comprise additional phosphate or an additional source of phosphate, and wherein the composition does not comprise fluoride.

In an eighth aspect there is provided a composition according to the fifth, sixth or seventh aspects for use in the treatment or prevention of dental caries or a dental condition involving dental demineralisation by administering the composition to the mouth of a subject.
In one embodiment, a composition for use in remineralisation or prevention of demineralisation comprises calcium.
In one embodiment, a composition for use in remineralisation or prevention of demineralisation does not comprise fluoride. Suitably therefore a preferred embodiment of the invention is a composition according to the fifth, sixth or seventh aspects which does not comprise fluoride, for use in the treatment or prevention of dental caries or a dental condition involving dental demineralisation by administering the composition to the mouth of a subject.
In one embodiment, the dental condition involving dental demineralisation is a lesion, suitably an erosive lesion.

In a ninth aspect there is provided a method of treating or preventing a dental caries or a dental condition involving dental demineralisation by administering a composition according to the fifth, sixth or seventh aspects to the mouth of a subject.
In one embodiment, a composition for use in remineralisation or prevention of demineralisation does not comprise fluoride. Suitably therefore a preferred embodiment of the invention is a method of treating or preventing a dental caries or a dental condition involving dental demineralisation by administering a composition according to the fifth, sixth or seventh aspects which does not comprise fluoride to the mouth of a subject.

In a tenth aspect there is provided packaging comprising a composition according to the fifth aspect, or a composition for use (optionally in a method) according to any of the first, second, third, fourth, sixth, seventh, eighth or ninth aspects.
Suitable compositions for any of the above aspects are discussed below. In some embodiments, the composition in any of the above aspects is an oral care composition, suitably according to the fifth aspect.
In some embodiments of the above aspects, the composition comprises osteopontin or phosphopeptides derived therefrom, and/or casein or phosphopeptides derived therefrom.
Suitably the compositions of any of the above aspects may or may not comprise fluoride. In some embodiments of the above aspects, the composition further comprises fluoride or a source of fluoride, such as monofluorophosphate. In some embodiments of the above aspects the composition does not comprise fluoride or a source of fluoride. Suitably, in some embodiments where the composition does not comprise additional phosphate or an additional source of phosphate, the composition further does not comprise fluoride or a source of fluoride
Suitably the compositions of any of the above aspects may or may not comprise calcium. In some embodiments of the above aspects, the composition further comprises calcium or a source of calcium. In some embodiments, the composition does not comprise calcium or a source of calcium. Suitably, in an embodiment where the composition is for use in remineralisation or preventing demineralisation of an oral surface, the composition comprises calcium or a source of calcium.
Suitably, unless otherwise stated, the compositions of any of the above aspects may comprise phosphate or may be phosphate free. In some embodiments of the above aspects, the composition further comprises an additional phosphate or an additional source of phosphate, such as phosphate buffers. In some embodiments of the above aspects the composition does not comprise additional phosphate or an additional source of phosphate.

In an eleventh aspect there is provided a method of manufacturing a composition according to any of the above aspects.
In one embodiment, the method of the eleventh aspect may comprise a ‘one pot’ method. Suitably the method may comprise the method described in WO2018/087532.

The present inventors have discovered oral care compositions which solve the above-mentioned problems of (i) staining and/or (ii) demineralisation.

With regard to staining, the present inventors have discovered that a composition comprising a phosphopeptide is effective at preventing staining of oral surfaces such as teeth. Compositions comprising phosphopeptides have traditionally been used for remineralisation or prevention of demineralisation of oral surfaces and inhibiting biofilms or bacteria which lead to such demineralisation. Use of these compositions for preventing staining has not been described before. Traditionally, other whitening agents have been added to oral care compositions to combat staining, such as particulates in toothpaste or mouthwash, and bleaching chemicals in whitening gels or strips. Advantageously, the compositions of the present invention need only contain phosphopeptides to achieve a reduction or prevention of staining, which are not damaging to the oral environment or toxic. Furthermore, these compositions can provide the dual effect of preventing staining and preventing demineralisation. In addition to the stain prevention effect of phosphopeptides discovered by the inventors, they have further found that use of a composition comprising a phosphopeptide after using a whitening agent on an oral surface prolongs the effect of the whitening agent, thereby preventing staining of the oral surface for longer.

With regard to preventing demineralisation and remineralisation, the inventors have further discovered that typical compositions containing phosphopeptides, need not contain additional phosphate to achieve such remineralisation. Prior oral care compositions containing phosphopeptides typically contain further phosphate in the form of buffers to aid in remineralizing an oral surface such as teeth. It is believed that phosphate ions are necessary to bind to the enamel surface and enhance remineralisation by changing the position of the equilibrium to favour the apatite crystalline form. However, these ions are prone to forming insoluble salts upon mixing in aqueous solution, and have been difficult to incorporate into oral care compositions. Prior solutions to this problem have been to mix ionic solutions comprising phosphate, calcium and fluoride with phosphopeptides which stabilise soluble amorphous calcium phosphate within the composition. However, the inventors have found that the phosphopeptides can achieve this effect alone without additional phosphate. Therefore, the production of the compositions of the invention is much easier. Furthermore, the inventors have surprisingly found that remineralisation is at least sustained, and often improved, when using a composition of the invention that does not comprise additional phosphate. The inventors have demonstrated that consistent daily use of the substantially phosphate-free compositions of the invention can provide surprisingly high levels of rapid and progressive remineralisation back to sound enamel. Surprising levels of over 80% remineralisation are achieved in the examples contained herein.

Further features and embodiments of the above aspects are defined hereinbelow under headed sections. Any feature in any section may be combined with any aspect or embodiment in any workable combination.

Compositions:

The compositions of the present invention are suitably oral care compositions, which may be for use in preventing staining of an oral surface such as teeth and/or for use in remineralizing an oral surface such as organic and inorganic mineralized tooth surfaces.

Suitably the compositions of the invention may be a fluid or a solid. Suitably the compositions of the invention may be a fluid selected from a liquid or a colloid. Suitably, the compositions may be an oral spray; a mouthwash; a toothpaste, cream or gel; a serum; chewing gum, powder or granules; wafer tabs; delivery strips; tablets; capsules; or the like.

Suitably any of the components described herein may be mixed in any workable combination to form a composition of the invention falling within one of the above aspects. It will be appreciated that certain components such as sources of phosphate are not intended for compositions for use in the fifth, sixth or seventh aspects, but may well be present in compositions for use in the first, second, third or fourth aspects.

Suitably, the composition is an aqueous composition, suitably an aqueous fluid. Suitably, the composition may therefore comprise an aqueous medium. Suitably the composition may comprise about 20%, about 25%, about 30%, about 35%, about 40%, about 45%, about 50%, about 55%, about 60%, about 65%, about 70%, about 75%, about 80%, about 90% by weight of water. Suitably the composition may comprise between about 20% to about 75% by weight of water.

Suitably, in one embodiment, the composition may be liquid. Suitably an aqueous liquid. Suitably in such embodiments, the composition comprises at least 50% by weight of water. Suitably in such embodiments, the composition may be a mouth wash or an oral spray.

Suitably, in an alternative embodiment, the composition may be a colloid. Suitably a colloidal composition may encompass a gum, gel, tablet or paste, for example. Suitably in such embodiments, the composition comprises less than 25% by weight of water. Suitably in such embodiments, the composition may be a toothpaste.

Suitably, in some embodiments, the composition may comprise phosphate, fluoride and/or calcium, or a source thereof, unless otherwise stated. Suitable sources of these components are described elsewhere herein.

Suitably the composition may further comprise other components, suitably the other components are liquid or soluble. Suitably the other components may be selected from: one or more of alcohol(s), humectant(s), surfactant(s), preservative(s), flavouring agent(s), sweetening agent(s), colouring agent(s), anti-caries agent(s), buffer(s), acid(s), base(s), whitening agent(s), thickener(s), and anticalculus agent(s).

The amounts of the various components of the compositions of the present invention can of course be determined by the person skilled in the art. Suitably the amounts of the various components making up a composition add up to 100% w/w of the composition.

Suitably, the composition may comprise a pH buffering agent (or buffer). Various pH buffering agents are well-known to the skilled person. Exemplary buffers include, but are not limited to, phosphate buffers, Tris (tris(hydroxymethyl)aminomethane) buffers, and sodium bicarbonate. In one embodiment, the buffer is sodium bicarbonate.

Suitably, the pH buffering agent maintains the composition at a pH of above 7, suitably in the range of from pH 7 to 9, suitably in the range of pH 7.1 to 8.5, suitably in the range of pH 7.2 to 8. Suitably said pH is maintained for a period of storage at room temperature of at least 6 weeks, 3 months, 6 months, suitably at least 1 year. Suitable buffering agents to achieve this will be apparent to the skilled person and their suitability for purpose can be readily determined experimentally. In one embodiment, the pH of the composition is stable for at least 10 months.

Suitably the initial pH of the composition may be adjusted by the addition of hydrogen ions (acid) or hydroxide ions (base), as required. Any physiologically compatible or acceptable acid or base may typically be used, e.g. hydrochloric acid (HCl) and sodium hydroxide (NaOH). Suitably once the desirable pH is reached, the buffer acts to maintain the pH.

Suitably the composition may or may not comprise one or more alcohols. Exemplary alcohols include, but are not limited to, ethanol, or isopropanol. Suitably, the compositions of the invention may comprise a sweetener alcohol, as explained below, however suitably no other additional alcohol is present. In one embodiment, therefore, the compositions of the invention do not contain ethanol or isopropanol.

Suitably, where additional alcohol is present, the weight ratio of water to alcohol is in the range of from about 1:1 to about 20:1. Suitably, the total amount of water-alcohol mixture in this type of preparation is typically in the range of from about 70 to about 99.9% by weight of the preparation. Suitably the concentration of the additional alcohol may be between 1-99%.

Suitably the composition may comprise one or more sweeteners. Suitably the sweeteners may also be alcohols. Alternatively the sweeteners may be natural or synthetic sugars such as saccharine. Suitably the composition may comprise both saccharine and an alcohol sweetener. Exemplary alcohol sweeteners include, but are not limited to, xylitol or mannitol. In one embodiment the sweetener is xylitol, suitably when the composition is a liquid such as a mouthwash or oral spray. In one embodiment, the sweetener is mannitol, suitably when the composition is a colloid, such as toothpaste.

Suitably the composition may comprise a whitening agent. Suitably the whitening agent may be chemical or abrasive. Exemplary chemical whitening agents include, but are not limited to; sodium bicarbonate or hydrogen peroxide. Exemplary abrasive whitening agents include, but are not limited to; microparticles such as silica, or charcoal. In one embodiment, the whitening agent is Zeofree 113 microparticles, suitably when the composition is a colloid such as toothpaste.

Suitably the composition may comprise a thickener. Examples of thickeners include, but are not limited to, silica or xanthan gum. In one embodiment, the thickening agent comprises Zeofree 153 microparticles and xanthan gum, suitably when the composition is a colloid such as toothpaste.

Suitably the composition may comprise a flavouring agent. Suitably the flavouring agent comprises a number of different chemicals which may be natural or synthetic such as sugars, oils, esters, and the like. In one embodiment, the flavouring agent may comprise a mixture of saccharine, tego betain, and flavour oil. An exemplary flavouring agent may comprise the following (values provided as per amounts in the final composition):
Sodium methyl paraben; suitably in an amount of about 0.02% w/w
Phenoxyethanol; suitably in an amount of about 0.2% w/w
Saccharine; suitably in an amount of about 0.08% w/w
Tego betain; suitably in an amount of about 0.6% w/w
Water; suitably in an amount of about 6.3% w/w, suitably wherein the water is deionised water
Flavour oil; suitably in an amount of about 0.5% w/w

Suitably the flavour oil may comprise a natural or synthetic flavouring. Suitably the flavour oil may comprise a herb or plant extract. Suitably the flavour oil may comprise a mint flavour.

Suitably the composition may comprise a preservative. Examples of preservative include Sodium methyl paraben and phenoxyethanol. Suitably the preservative may comprise sodium methyl paraben; suitably in an amount of about 0.02% w/w and Phenoxyethanol; suitably in an amount of about 0.2% w/w.

Suitably, the composition may comprise any of the following components: water, buffer(s), a source of calcium ions, a source of phosphate ions, a phosphopeptide, a source of fluoride ions, a flavouring, a preservative, a sweetener, an acid, a whitening agent, a thickener. Suitably, the composition comprises at least the following components: water, buffer(s), a source of calcium ions, a phosphopeptide. In one embodiment, the composition comprises the following components: water, buffer(s), a source of calcium ions, a phosphopeptide, a source of fluoride ions, a flavouring and preservative, a sweetener, an acid. In one embodiment, the composition comprises the following components: water, buffer(s), a source of calcium ions, a phosphopeptide, a source of fluoride ions, a flavouring and preservative, a sweetener, an acid, a whitening agent, a thickener. Suitably, in some embodiments, the composition does not comprise fluoride. Suitably, in some embodiments, the composition does not comprise phosphate.

In some embodiments of the present invention, the components of the composition may individually be provided in the following amounts, or in any combination:
Water—from about 20% to about 99% by weight; suitably from about 23% to about 66% by weight;
A buffer—from about 1% to about 20% by weight; suitably from about 1% to about 15% by weight;
A source of calcium ions if present (e.g. a soluble calcium salt or other options as discussed herein)—from about 0.1% to about 15% by weight; suitably from about 0.1% to about 5% by weight;
A source of phosphate ions if present (e.g. a soluble phosphate salt or other options as discussed herein)—from about 0.2% to about 15% by weight; suitably from about 0.5% to about 5% w/v, suitably from about 0.7% to about 2% by weight, e.g. from 0.8 to 1.2% by weight.
A phosphopeptide—from about 0.5% to about 15% w/v; suitably from about 1% to about 10% by weight, suitably from about 1.5% to about 5% by weight, e.g. from 2 to 4% by weight.
A source of fluoride ions if present (e.g. a soluble fluoride salt such as monofluorophosphate as discussed herein)—from about 0.01% to about 3%; suitably from about 0.05% to about 1.5% by weight.

In some embodiments, the compositions may further comprise any of the following components alone, or in any combination:
A flavouring, preservative and/or other ingredients from about 0% to about 70% by weight; suitably from about 0% to about 20% by weight; suitably from about 0% to about 10% by weight;
A sweetener (e.g. a sweetener alcohol such as mannitol or xylitol)— from about 0.1% to about 20% (w/w); suitably from about 0.1% to about 10% (w/w);
An acid (e.g. HCl)—from about 5-40% (w/w); suitably from about 10-35% (w/w);
A whitening agent (e.g. abrasive silica)—from about 1-20% (w/w); suitably from about 5-10% (w/w);
A thickener (e.g. xanthan gum and/or silica)—from about 0.1-20% (w/w), suitably from about 0.5-15% (w/w)

As noted above, in one embodiment, the composition is an oral spray or mouthwash. Exemplary mouthwash and spray formulations are set out below.

Mouth washes and mouth sprays according to the present invention can suitably include the following exemplary components by weight:
water (suitably from about 45% to about 95%),
ethanol (suitably from about 0% to about 25%),
humectant(s) (suitably from about 0% to about 50%),
surfactant(s) (suitably from about 0.01% to about 7%),
flavouring agent(s) (suitably from about 0.04% to about 2%),
sweetening agent(s) (suitably from about 0.1% to about 8%),
colouring agent(s) (suitably from about 0% to about 0.5%),
xylitol (suitably from about 0% to about 8%),
anti-caries agent(s), including but not limited to stabilised calcium phosphate and fluoride (suitably from about 0.001% to 10%), and optionally
an anti-calculus agent (suitably from about 0% to about 13%).

One exemplary composition of the present invention comprises:
A phosphopeptide; suitably in an amount of about 3% w/w, suitably wherein the phosphopeptide is OPN-10
A buffer; suitably in an amount of about 15% w/w, suitably of a 1M solution, suitably wherein the buffer is sodium bicarbonate
Water; suitably in an amount of about 54% to 66% w/w, suitably wherein the water is deionised water
A source of calcium; suitably in an amount of about 3% w/w, suitably 2.9% w/w, suitably wherein the source of calcium is a calcium chloride solution, suitably wherein the calcium chloride solution is about 1M
Optionally, a source of fluoride suitably in an amount of about 0.4% to 0.5% w/w, suitably 0.4% w/w, suitably wherein the source of fluoride is monofluorophosphate.
A sweetener; suitably in an amount of about 5% w/w, suitably wherein the sweetener is xylitol
Optionally an acid; suitably in an amount of about 11% w/w, suitably wherein the acid is hydrochloric acid, suitably wherein the hydrochloric acid is about 1M
A flavouring and preservative agent; suitably in an amount of about 8% w/w, suitably 7.7% w/w, suitably wherein the agent comprises a mixture of sodium methyl paraben, phenoxyethanol, saccharine, tego betain, and flavour oil.

Suitably such an exemplary composition is a liquid. Suitably such an exemplary composition is a mouth wash or an oral spray. Suitably such an exemplary composition may be known as ‘MOL’.

In some embodiments, the exemplary composition above does not comprise a source of fluoride. Suitably it does not comprise monofluorophosphate. Suitably, in some embodiments, the composition does not comprise phosphate.

In some embodiments, the exemplary composition above does not comprise an acid. Suitably it does not comprise hydrochloric acid.

Suitably, water is added to make up the final composition to 100% w/w. Suitably, in the absence of any component, further water is added instead. In one embodiment, the water is in an amount of 55% w/w. In one embodiment the water is in an amount of 55.4% w/w, suitably in such an embodiment, the composition does not comprise a source of fluoride. In one embodiment, the water is in an amount of 66.4% w/w, suitably in such an embodiment, the composition does not comprise a source of fluoride or an acid.

Suitably the MOL composition may be used in any aspect of the invention, for remineralisation and/or prevention of demineralisation, or for prevention and/or reduction of staining, or for prolonging whitening. In one embodiment the MOL composition may be for use in the first aspect; for preventing or reducing staining of an oral surface.

Another exemplary composition of the present invention comprises:
A phosphopeptide; suitably in an amount of about 3% w/w; suitably wherein the phosphopeptide is OPN-10
A buffer; suitably in an amount of about 2% w/w, suitably wherein the buffer is sodium bicarbonate
A whitening agent; suitably in an amount of about 6% w/w, suitably wherein the whitening agent comprises an abrasive silica, for example zeofree 113
Water; suitably in an amount of about 23 to 25% w/w, suitably wherein the water is deionised water
An acid; suitably in an amount of about 34% w/w, suitably wherein the acid is hydrochloric acid, suitably wherein the hydrochloric acid is about 1M
A source of calcium; suitably in an amount of about 0.5% w/w, suitably wherein the source of calcium is calcium chloride
Optionally a source of fluoride, suitably in an amount of about 1% w/w, suitably 1.1% w/w, suitably wherein the source of fluoride is monofluorophosphate
A sweetener; suitably in an amount of about 9% w/w, wherein suitably the sweetener is mannitol
A thickener; suitably in an amount of about 13% w/w, suitably 12.8% w/w, wherein suitably the thickener comprises a thickening silica and xanthan gum, for example Zeofree 153 and xanthan gum
A flavouring and preservative agent; suitably in an amount of about 8% w/w, suitably 7.7% w/w, suitably wherein the agent comprises a mixture of sodium methyl paraben, phenoxyethanol, saccharine, tego betain, and flavour oil.

Suitably such an exemplary composition is a colloid. Suitably such an exemplary composition is a toothpaste. Suitably such an exemplary composition may be known as ‘MON’.

In some embodiments, the exemplary composition above does not comprise a source of fluoride. Suitably it does not comprise monofluorophosphate. Suitably, in some embodiments, the composition does not comprise phosphate.

Suitably, water is added to make up the final composition to 100% w/w. Suitably, in the absence of any component, further water is added instead. In one embodiment, the water is in an amount of 23.9% w/w. In one embodiment, the water is in an amount of 25% w/w, suitably in such an embodiment, the composition does not comprise a source of fluoride.

Suitably the MON composition may be used in any aspect of the invention, for remineralisation and/or prevention of demineralisation, or for prevention and/or reduction of staining, or for prolonging whitening. In one embodiment, the MON composition may be for use in the sixth aspect, for use in remineralisation or prevention of demineralisation of an oral surface.

Phosphopeptides:

The term “phosphopeptides” is used herein to describe phosphorylated polypeptides in a general sense. The term phosphopeptides is used interchangeably with phosphoprotein unless the context dictates otherwise. A range of phosphopeptides that can be used in the present invention are well-known in the art, and several are described in detail below.

Phosphopeptides that are able to interact with and stabilise calcium phosphate complexes are of particular interest, though it will be noted that in the present invention the phosphopeptides need not perform such a role, e.g. when additional phosphate or an additional source of phosphate is absent.

In particular, mention can be made of osteopontin or phosphopeptides derived therefrom, and casein or phosphopeptides derived therefrom. These two phosphoproteins and their phosphopeptides have been extensively discussed in the literature in respect of forming stabilised calcium phosphate complexes. However, there are other phosphopeptides which can form stabilised calcium phosphate complexes, such as phosvitin (Swiss-Prot Accession No P67869), fetuin A (FETUA) (Swiss-Prot Accession No P02765), proline-rich basic phosphoprotein 4 (PRB4) (Swiss-Prot Accession No PI 0163), matrix Gla protein (MGP) (Swiss-Prot Accession No P08493), secreted phosphoprotein 24 (SPP-24) (Swiss-Prot Accession No Q13103), Riboflavin Binding Protein (Swiss-Prot Accession No P02752), integrin binding sialophosphoprotein II (IBSP-II) (Swiss-Prot Accession No P21815), matrix extracellular bone phosphoglycoprotein (MEPE) (Swiss-Prot Accession No Q9NQ76), dentin matrix acidic phosphoprotein 1 (OMP1) (Swiss-Prot Accession No Q13316), human beta-casein, bovine beta-casein, and isoforms or phosphopeptides derived therefrom. Moreover, there are potentially a wide range of synthetic phosphopeptides that can be used in the present invention.

Thus, suitable phosphopeptides may be from any source and take a number of forms. For example, suitable phosphopeptides include full length phosphoproteins, or smaller phosphopeptides derived therefrom that may be naturally occurring or may be formed or isolated by tryptic or chemical (e.g. alkaline hydrolysis) digestion of such phosphoproteins, or obtained by chemical or recombinant synthesis. The phosphopeptide may be osteopontin or casein, or may be derived from osteopontin, casein, or other phosphoamino acid rich proteins such as phosvitin (Swiss-Prot Accession No P67869), fetuin A (FETUA) (Swiss-Prot Accession No P02765), proline-rich basic phosphoprotein 4 (PRB4) (Swiss-Prot Accession No PI 0163), matrix Gla protein (MGP) (Swiss-Prot Accession No P08493), secreted phosphoprotein 24 (SPP-24) (Swiss-Prot Accession No Q13103), Riboflavin Binding Protein (Swiss-Prot Accession No P02752), integrin binding sialophosphoprotein II (IBSP-II) (Swiss-Prot Accession No P21815), matrix extracellular bone phosphoglycoprotein (MEPE) (Swiss-Prot Accession No Q9NQ76), dentin matrix acidic phosphoprotein 1 (OMP1) (Swiss-Prot Accession No Q13316), human beta-casein, bovine beta-casein, and isoforms or phosphopeptides derived therefrom.

In one embodiment, phosphopeptides obtained by enzymatic (e.g. tryptic) digest of osteopontin or casein are used in the present invention.

Osteopontin (OPN) is a protein that can be obtained from milk. For example, bovine OPN can be isolated by anion exchange chromatography from e. g. acid whey at pH 4.5 as described by the patent applications WO 01/497741 A2, WO 02/28413, WO 2012/117,119 or WO 2012/117,120. An OPN purity of up to 90-95% can be obtained. The present invention can use naturally occurring fragments or peptides derived from OPN by proteolytic cleavage in the milk, or genesplice-, phosphorylation-, or glycosylation variants as obtainable from the method proposed in, for example, WO 01/49741 and WO2013/144247. OPN can be derived from milk from any milk producing animals, such as cows, humans, camels, goats, sheep, dromedaries and llamas. OPN from bovine milk is typically preferred due to availability and characterisation in the literature. OPN is present in bovine milk, both in the form of full length bovine OPN (e.g. position 17-278 of Swiss-Prot Accession No P31096, or a peptide having at least 95% sequence identity with position 17-278 of Swiss-Prot Accession No P31096) and in the form of a long N-terminal fragment of full length bovine OPN (e.g. position 17-163 of Swiss-Prot Accession No P31096, or a peptide having at least 95% sequence identity with position 17-163 of Swiss-Prot Accession No P31096), see e.g. Bissonnette et al., Journal of Dairy Science Vol. 95 No. 2, 2012). Full length OPN is an acidic, highly phosphorylated, sialic acid rich, calcium binding protein. Full length osteopontin binds 28 moles of phosphate and about 50 moles of Ca per mole. The use of OPN to form calcium phosphate complexes is discussed extensively in, for example, WO2013/144247, particularly but not exclusively in respect of their use to treat biofilm related diseases.

In some embodiments of the invention, the OPN or phosphopeptides derived therefrom may be substantially pure full length bovine OPN, it may be a substantially pure, long N-terminal fragment of full length bovine OPN, and it may be a mixture comprising full length bovine OPN and the long N-terminal fragment of full length bovine OPN. Such a mixture may for example contain full length bovine OPN in an amount of 5-40% (w/w) relative to the total amount of OPN and the long n-terminal fragment of full length bovine OPN in an amount of 60-95% (w/w) relative to the total amount of OPN.

In one embodiment, the compositions of the invention comprise phosphopeptides derived from OPN (e.g. by the cleavage of OPN, such as by tryptic or chemical (e.g. alkaline hydrolysis) digestion of OPN). In one embodiment, the compositions of the invention comprise OPN-derived phosphopeptides sold commercially as Lacprodan® OPN-10. OPN-10 is available commercially from Arla Foods Ingredients (Arla Foods Ingredients Group P/S, Sønderhøj 10-12, 8260 Viby J, Denmark), and contains fractionated osteopontin from bovine milk.

Casein and casein-derived phosphopeptides are discussed at length in WO 98/40406 and WO 2006/135982, and these phosphopeptides can also suitably be used in the present invention.

As discussed in WO 2006/135982, CPP can form a colloidal complex with amorphous calcium phosphate, where the core particles aggregate to form large (e.g. 100 nm) colloidal particles suspended in water. It is believed that this general method of stabilisation of calcium phosphate also occurs for other phosphoproteins. Without wishing to be bound by theory, the phosphopeptide seems to bind to an amorphous calcium phosphate (ACP) cluster to produce a metastable solution in which growth of ACP to a size that initiates nucleation and precipitation is prevented.

Suitably, phosphopeptides comprising the motif Ser(P)-Ser(P)-Ser(P)-Glu-Glu, which is present in casein phosphopeptides may be used in the present invention. However, phosphopeptides comprising other sequence motifs rich in phosphoamino acids are also of use in the present invention.

Suitably, casein-derived phosphopeptides comprising the sequences αₛ₁(59-79), β (1-25), αₛ₂(46-70) and αₛ₁(1-21), as set out in WO 98/40406 and WO 2006/135982, may be used in the present invention. Additional flanking sequences surrounding these core sequences may be present, in which case they can be wild type sequences or may optionally be modified by deletion, addition or conservative substitution of one or more residues.

Accordingly, in embodiments of the present invention, the phosphopeptide comprises osteopontin or phosphopeptides derived therefrom, or casein or phosphopeptides derived therefrom. In some embodiments, the calcium phosphate-stabilising agent comprises osteopontin-derived phosphopeptides or casein-derived phosphopeptides.

Alternatively or additionally, the phosphopeptide comprises one or more phosphoproteins selected from the group consisting of phosvitin (Swiss-Prot Accession No P67869), fetuin A (FETUA) (Swiss-Prot Accession No P02765), proline-rich basic phosphoprotein 4 (PRB4) (Swiss-Prot Accession No PI 0163), matrix Gla protein (MGP) (Swiss-Prot Accession No P08493), secreted phosphoprotein 24 (SPP-24) (Swiss-Prot Accession No Q13103), Riboflavin Binding Protein (Swiss-Prot Accession No P02752), integrin binding sialophosphoprotein II (IBSP-II) (Swiss-Prot Accession No P21815), matrix extracellular bone phosphoglycoprotein (MEPE) (Swiss-Prot Accession No Q9NQ76), dentin matrix acidic phosphoprotein 1 (OMP1) (Swiss-Prot Accession No Q13316), human beta-casein, bovine beta-casein, and isoforms or phosphopeptides derived therefrom.

For the avoidance of doubt, it should be noted that in embodiments of the present invention, the compositions can comprise a mixture of different phosphopeptides. For example, the composition may comprise a mixture of different phosphopeptides derived from a single phosphoprotein (e.g. casein or OPN). Alternatively, the calcium phosphate-stabilising agent may comprise a mixture of different phosphoproteins (e.g. a mixture of casein and OPN, or other different phosphoproteins) and/or phosphopeptides derived from a mixture of more than one different phosphoprotein (e.g. a mixture of phosphopeptides derived from both casein and OPN). In many cases, the composition will comprise a heterogeneous mixture of phosphopeptides obtained by the cleavage of a naturally occurring protein, such as OPN or casein.

Stabilised Calcium Phosphate Complexes:

A “stabilised calcium phosphate complex” is a complex comprising calcium, phosphates and a calcium phosphate-stabilising agent (usually a phosphopeptide). The stabilised calcium phosphate complex is typically soluble or at least metastable in the liquid medium in which it is contained, i.e. the liquid composition. Calcium phosphate-stabilising agents (such as phosphopeptides) are able to bind to calcium phosphate complexes and prevent them from precipitating. In particular, amorphous calcium phosphate complexes can be stabilised in a form in which they remain soluble (or metastable) and are able to release calcium and phosphate.

Without wishing to be bound by theory, small amounts of phosphopeptide stabilised calcium phosphate complexes may be formed upon administration to the mouth of a suitable liquid composition containing a phosphopeptide and a source of calcium ions. Stabilised calcium phosphate complexes may be formed on interaction of exogenous calcium ions with phosphate ions present in saliva of the mouth.

Nonetheless, it is remarkable how the compositions of some of the aspects of the invention that do not comprise additional phosphate or an additional source of phosphate can still achieve high levels of remineralisation.

A “calcium phosphate-stabilising agent” is an agent that is capable of binding to and stabilising calcium phosphate in a stabilised calcium phosphate complex. Suitably, the calcium phosphate may be stabilised as amorphous calcium phosphate. Suitable calcium phosphate-stabilising agents include phosphopeptides, as explained above. Suitable phosphopeptides are defined herein.

Suitably compositions of certain aspects of the present invention do not comprise a stabilised calcium phosphate complex. Suitably compositions of certain aspects of the present invention do not comprise amorphous calcium phosphate. Suitably in aspects or embodiments where the composition does not comprise additional phosphate or an additional source of phosphate, suitably the composition does not comprise a calcium phosphate complex such as amorphous calcium phosphate.

Suitably, these compositions may comprise a negligible amount of a stabilised calcium phosphate complex. By a negligible amount, it is meant that these compositions comprise less than 1% w/w of a stabilised calcium phosphate complex, suitably less than 0.9% w/w, suitably less than 0.8% w/w, suitably less than 0.7% w/w, suitably less than 0.6% w/w, suitably less than 0.5% w/w, suitably less than 0.4% w/w, suitably less than 0.3% w/w, suitably less than 0.2% w/w, suitably less than 0.1% w/w. In one embodiment, the compositions that do not comprise additional phosphate or an additional source of phosphate comprise less than 0.1% w/w of a stabilised calcium phosphate complex.

Calcium and Sources of Calcium:

The compositions of the invention may comprise calcium or a source of calcium.

In one embodiment, the compositions of the sixth and seventh aspects for use in remineralisation or prevention of demineralisation comprise calcium or a source of calcium.

Suitably the compositions of the first or second aspects for use in prevention of staining, or the third aspect for prolonging whitening, may also comprise calcium or a source of calcium.

Calcium and sources of calcium as used herein refers to any suitable source of calcium ions. A source of calcium ions should be able to dissolve in the liquid medium to release calcium ions. Suitably the source of calcium ions may be a soluble calcium salt. Suitably the source of calcium ions has a solubility of 5 g per 100 ml of liquid medium or higher, 10 g per 100 ml of liquid medium or higher, or 50 g per 100 ml of liquid medium or higher. The source of calcium ions can be provided in solid form or be dissolved in a suitable liquid.

One particularly suitable source of calcium ions is calcium chloride, but the person skilled in the art can select many other suitable sources of calcium ions.

In one embodiment, the source of calcium is calcium chloride. Suitably the calcium chloride solution has a concentration of about 1M.

When discussing the solubility of a composition herein, it is meant solubility at 25° C. (and otherwise standard conditions) in the relevant liquid medium used in the method. Typically, this medium will be aqueous, and in some cases will be water. It will be appreciated that the solubility of a given composition will vary depending on the relevant medium being used, e.g. depending on its polarity, but in the context that is entirely appropriate as the compositions such as calcium or phosphate salts are preferably soluble in the relevant medium being used. However, given that in the compositions of the present invention the medium is typically aqueous, it may be more convenient and simple to define the solubility in terms of solubility in water. Accordingly, solubility of the source of calcium ions is suitably of 5 g per 100 ml of water or higher, 10 g per 100 ml of water or higher, or 50 g per 100 ml of water or higher.

Phosphate and Source of Phosphate:

In some aspects, the compositions of the invention may comprise phosphate or a sources of phosphate. However preferably the compositions of the invention do not comprise additional phosphate or an additional source of phosphate.

By ‘additional’ phosphate or source of phosphate it is meant that the composition does not comprise any phosphate or source of phosphate other than the phosphopeptide and optionally monofluorophosphate of the composition.

Suitably, therefore, in one embodiment, the compositions do not comprise phosphate or a source of phosphate other than phosphorylated OPN and/or monofluorophosphate.

Therefore, in one embodiment, the composition of the invention or for use in the invention comprises a phosphopeptide and optionally monofluorophosphate, and does not comprise additional phosphate or an additional source of phosphate. Typical additional sources of phosphate may comprise phosphate buffers, suitably these are not present in the compositions.

In some embodiments, the compositions of the invention do not comprise additional phosphate ions or an additional source of phosphate ions. In some embodiments, the compositions of the invention do not comprise additional free phosphate. In some embodiments, the compositions of the invention do not comprise any exogenous free phosphate. In some embodiments, the compositions of the invention do not comprise additional free phosphate ions. In some embodiments, the compositions of the invention do not comprise exogenous free phosphate ions.

By ‘exogenous’ it is meant phosphate that is not derived from or comprised within a compound that is part of the compositions of the invention. Suitably it is meant phosphate that is not derived from or comprised within the phosphopeptide and optionally the monofluorophosphate of the compositions of the invention.

Suitably such compositions of the invention may be regarded as substantially phosphate-free. Suitably these compositions may comprise trace amounts of phosphate due to other components present in the composition, but suitably these compositions do not comprise any significant amount of phosphate, or a source of phosphate. Suitably, in particular, the compositions do not comprise phosphate buffers.

Suitably, therefore, the composition comprises a low amount of phosphate. Suitably a negligible amount of phosphate, for example, suitably less than 15% w/w phosphate, suitably less than 14% w/w phosphate, suitably less than 13% w/w phosphate, suitably less than 12% w/w phosphate, suitably less than 11% w/w phosphate, suitably less than 10% w/w phosphate, suitably less than 9% w/w phosphate, suitably less than 8% w/w phosphate, suitably less than 7% w/w phosphate, suitably less than 6% w/w, suitably less than 5% w/w, suitably less than 4% w/w, suitably less than 3% w/w, suitably less than 2% w/w. In one embodiment, the compositions that do not comprise phosphate or a source of phosphate comprise less than 7% w/w phosphate. Suitably, as noted above, a negligible amount of phosphate may come from components of the compositions such as monofluorophosphate and the phosphopeptide such as phosphorylated OPN.

Suitably a composition that does not comprise additional phosphate or an additional source of phosphate comprises less than 100 mM, less than 90 mM, less than 80 mM, less than 70 mM, less than 60 mM, less than 55 mM, less than 50 mM, less than 45 mM, less than 40 mM, less than 35 mM, less than 30 mM, less than 25 mM, less than 20 mM, less than 15 mM phosphate, less than 10 mM phosphate, less than 5 mM phosphate. Suitably a composition that does not comprise additional phosphate or an additional source of phosphate comprises less than 49 mM phosphate. Suitably a composition that does not comprise additional phosphate or an additional source of phosphate comprises less than 23 mM phosphate.

Suitably the concentration of phosphate in the composition is determined by the David Smillie calculation based on the degree of phosphorylation of any compounds in the composition. An exemplary calculation of the concentration of phosphate in a composition comprising phosphopeptide and monofluorophosphate according to the preferred embodiments of the invention is as follows:

$$3 \% OPN = 30 g / L$$

$$7 \% phosphorylation ( Davids figgure ) = 2.1 g phosphate$$

$$Molecular weight of phospate = 94.97 g / mol$$

$$\frac{1 M}{94.97 g} \times 2.1 g = 0.022 M$$

$$3.8 g MFP in 1 L Toothboost .$$

$$Molecular weight of MFP = 143.95 g / mol$$

$$\frac{1 M}{143.95 g} \times 3.8 g = 0.02639 M$$

$$0.022 M + 0.02639 M = 48.39 mM$$

In one embodiment, a composition that does not comprise additional phosphate or an additional source of phosphate comprises less than 48.5 mM phosphate. Suitably in such an embodiment, the composition still comprises a phosphopeptide and monofluorophosphate. Suitably the composition comprises about 3% w/w phosphorylated OPN.

In one embodiment, a composition that does not comprise additional phosphate or an additional source of phosphate comprises less than 22.5 mM phosphate. Suitably in such an embodiment, the composition still comprises a phosphopeptide, but does not comprise monofluorophosphate. Suitably the composition comprises about 3% w/w phosphorylated OPN.

Suitably the compositions for prevention of staining or reducing staining may optionally comprise additional phosphate or an additional source of phosphate. Suitably, therefore, in the first, second and third aspects, the composition may comprise phosphate or a source of phosphate. However, in some embodiments, such compositions may not comprise additional phosphate or an additional source of phosphate.

Suitably in the fifth, sixth aspects and seventh aspects, the composition does not comprise additional phosphate or an additional source of phosphate as defined hereinabove.

‘Phosphate’ and a ‘source of phosphate’ as used herein refers to any suitable source of phosphate ions. If present in the composition, the source of phosphate ions should be able to dissolve in a liquid medium to release phosphate ions. Suitably the source of phosphate ions may be a soluble phosphate salt. Suitably the source of phosphate ions has a solubility of 5 g per 100 ml of liquid medium or higher, 10 g per 100 ml of liquid medium or higher, 50 g per 100 ml of liquid medium or higher. The source of phosphate ions can be provided in solid form or be dissolved in a suitable liquid.

One particularly suitable source of phosphate ions is sodium phosphate (e.g. disodium hydrogen phosphate and/or trisodium phosphate), but the person skilled in the art can select many other suitable sources of phosphate ions.

As discussed above, given that in the compositions of the present invention the medium is typically aqueous, it may be more convenient and simple to define the solubility in terms of solubility in water. Accordingly, solubility of the source of phosphate ions is suitably of 5 g per 100 ml of water or higher, 10 g per 100 ml of water or higher, or 50 g per 100 ml of water or higher.

Fluoride and Sources of Fluoride:

The compositions of the invention may comprise fluoride or a source of fluoride. However suitably in many preferred embodiments of the invention, the compositions do not comprise fluoride.

Suitably the composition for remineralisation or prevention of demineralisation may comprise fluoride. Suitably the composition for prevention of staining may comprise fluoride. However, in some embodiments, the composition does not comprise fluoride or a source of fluoride. Fluoride and sources of fluoride as used herein refers to any suitable source of fluoride ions. A source of fluoride ions should be able to dissolve in the liquid medium to release fluoride ions. Suitably the source of fluoride ions may be a soluble fluoride salt. Suitably the source of fluoride ions has a solubility of 5 g per 100 ml of liquid medium or higher, 10 g per 100 ml of liquid medium or higher, or 50 g per 100 ml of liquid medium or higher. The source of fluoride ions can be provided in solid form or be dissolved in a suitable liquid.

Suitable sources of fluoride ions are sodium fluoride, tin fluoride, calcium fluoride, and monofluorophosphate, but the person skilled in the art can select many other suitable sources of fluoride ions.

In one embodiment, the source of fluoride is monofluorophosphate.

As discussed above, given that in the compositions of the present invention the medium is typically aqueous, it may be more convenient and simple to define the solubility in terms of solubility in water. Accordingly, solubility of the source of fluoride ions is suitably of 5 g per 100 ml of water or higher, 10 g per 100 ml of water or higher, or 50 g per 100 ml of water or higher.

In some embodiments, the compositions of the invention do not comprise fluoride or a source of fluoride. In some embodiments, the compositions of the invention do not comprise fluoride. Suitably such compositions of the invention may be regarded as substantially fluoride-free. Suitably, these compositions may comprise trace amounts of fluoride due to other components present in the composition, but suitably these compositions do not comprise any significant amount of fluoride, or a source of fluoride. Suitably fluoride-free compositions are regarded as those having a negligible amount of fluoride, suitably less than 1% w/w fluoride or a source of fluoride, suitably less than 0.9% w/w, suitably less than 0.8% w/w, suitably less than 0.7% w/w, suitably less than 0.6% w/w, suitably less than 0.5% w/w, suitably less than 0.4% w/w, suitably less than 0.3% w/w, suitably less than 0.2% w/w, suitably less than 0.1% w/w. In one embodiment, the compositions that do not comprise fluoride or a source of fluoride comprise less than 0.1% w/w fluoride.

In some embodiments, the compositions of the invention do not comprise monofluorophosphate. Suitably, in some embodiments, the composition is a liquid. Suitably, the composition may be a ‘MOL’ composition as described elsewhere herein. Suitably, in some embodiments, the composition is a colloid or paste. Suitably, the composition may be a ‘MON’ composition as described elsewhere herein.

Oral Surface:

The compositions of the present invention may be applied to an oral surface. Suitably for use in preventing staining, or prolonging whitening, and/or remineralisation and/or preventing demineralisation of the oral surfaces, or in the treatment or prevention of dental caries or a dental condition involving dental demineralisation.

Suitably the oral surface is a natural surface or a synthetic surface. Suitably the oral surface may be a hard surface or a soft surface. Suitably the hard surfaces may be natural or synthetic. Suitably the soft surfaces may be natural or synthetic.

Suitable hard surfaces include teeth, dentures, veneers, braces and the like. Suitably the hard surfaces are mineralized. Suitable hard mineralized surfaces include enamel, dentine or cementum. Suitably the enamel may be surface or subsurface enamel. Suitably the soft surfaces are not mineralized. Suitable soft surfaces include gums, tongue or orthodontic aligners and bands.

Suitable natural surfaces include teeth, gums, tongue. Suitably the synthetic surfaces include oral appliances, or oral accessories and prosthesis. Suitable synthetic surfaces may be formed of plastics or metal. Suitable oral appliances may include dental equipment and tools. Suitably oral accessories may include dentures, veneers, braces, aligners, orthodontic bands, retainers, bridges and the like.

Suitably in aspects where the composition is for use in the prevention of staining or prolonging whitening, the oral surface may be a soft or hard surface, and may be any natural or synthetic surface. In one embodiment, the oral surface may be a hard natural surface such as the enamel of a tooth. In one embodiment, the enamel may be surface enamel.

Suitably in aspects where the composition is for use in the prevention of demineralisation, the oral surface is a hard surface, and may be any natural or synthetic surface, suitably a mineralized surface. In one embodiment, the oral surface may be a hard natural mineralized surface such as the enamel of a tooth. In one embodiment, the enamel may be surface or subsurface enamel.

Suitably the oral surface may be located within a mouth. Suitably within a mouth of a subject. Suitably the subject may be a human or animal. Suitably therefore the uses and methods of the invention may be performed on a human or animal subject. In one embodiment, the subject is a human. In one embodiment, the subject is a domestic animal, such as a cat or a dog.

Suitably, the uses and methods of the invention comprise applying the compositions of the invention to an oral surface. Suitably applying the compositions of the invention to an oral surface may comprise administering the composition to the mouth of a subject. Suitably administering the composition to the mouth of a subject contacts the oral surfaces located within the mouth of the subject with the composition. Suitable programs of administration are described hereinbelow.

Prevention of Staining:

Some aspects of the present invention relate to use of compositions in preventing or reducing staining of an oral surface.

Suitably the compositions for preventing or reducing staining may or may not remineralise an oral surface. Suitably the compositions for preventing or reducing staining may or may not Inhibit bacteria, and therefore may or may not inhibit biofilm formation.

However, in one embodiment, the compositions for preventing or reducing staining are not for use in remineralizing an oral surface or preventing demineralisation of an oral surface. Similarly, in one embodiment, the compositions for preventing or reducing staining are not for use in removing bacteria, and therefore are not for use in removing biofilms.

Suitably the compositions for preventing or reducing staining may be dentifrice, liquid, colloids (e.g. toothpaste), or solids. Suitably the compositions for preventing or reducing staining are liquid compositions, suitably aqueous liquid compositions. Suitably the compositions for preventing or reducing staining may be a mouthwash or an oral spray. Suitably such liquid compositions have formulations as described elsewhere herein.

Suitably references to preventing or reducing staining as used herein may refer to protection of the oral surface from staining. Suitably the composition comprising a phosphopeptide may be for use in protection of an oral surface from staining.

Suitably the compositions for preventing or reducing staining may prevent or reduce staining by up to 50% compared to a non-treated oral surface. Suitably the compositions for preventing or reducing staining may prevent or reduce staining by 10%, 20%, 30%, 40% compared to a non-treated oral surface. Suitably the level of prevention or reduction in staining is measured using the method described in ‘In vitro evaluation of a novel 6% hydrogen peroxide tooth whitening product’ Andrew Joiner, Gopal Thakker. Journal of Dentistry (2004) 32, 19-25, and as described in the examples.

Suitably the oral surface may be a hard or soft surface. Suitably the oral surface may be a natural surface or a synthetic surface. Suitably in one embodiment, the oral surface is teeth, suitably the surface of teeth, suitably the enamel of teeth.

Suitably therefore, the compositions for preventing or reducing staining may prevent or reduce staining of teeth by up to 50% compared to a non-treated teeth. Suitably the compositions for preventing or reducing staining may prevent or reduce staining of teeth by 10%, 20%, 30%, 40% compared to a non-treated oral teeth.

Suitably, in another embodiment, the oral surface is an oral accessory, suitably the surface of an oral accessory, suitably a polymer or metal surface of an oral accessory.

Suitably therefore, the compositions for preventing or reducing staining may prevent or reduce staining of an oral accessory by up to 50% compared to non-treated oral accessories. Suitably the compositions for preventing or reducing staining may prevent or reduce staining of an oral accessory by 10%, 20%, 30%, 40% compared to a non-treated oral accessories.

Suitably the compositions of the invention are in general non-staining. Suitably the compositions of the invention do not stain oral surfaces.

Suitably the compositions for preventing or reducing staining may be applied to an oral surface at least once per day, suitably twice or three times per day. Suitably, when the oral surface is within the mouth of a subject, the compositions for preventing or reducing staining may be for administration to the subject at least once per day, suitably twice or three times per day, suitably to the mouth of the subject. Suitably for at least 5 days, at least 6 days, at least 7 days, at least 8 days, at least 9 days, at least 10 days, at least 11 days, at least 12 days, at least 13 days, at least 14 days, at least 15 days.

Prolonged Whitening

Some aspects of the present invention relate to use of compositions for prolonging the whiteness of an oral surface.

Suitably such compositions are those of the first or second aspects that may be used for prevention or reduction in staining of an oral surface. However, they may also be the composition of the third aspect of the invention.

Suitably the compositions of the invention may be used to prolong the whiteness of an oral surface after contacting with a whitening agent. Suitably, use in prolonging the effect of a whitening agent means that the compositions of the invention allow whitened oral surfaces to stay whiter for a longer period of time.

Suitably therefore, the compositions of the invention may be contacted with an oral surface after the surface is contacted with a whitening agent. Suitably the oral surface after whitening may be referred to as a ‘whitened oral surface’. Suitably the composition of the invention is contacted with the whitened oral surface after whitening.

Suitably therefore the compositions of the invention may be for use in prolonging the whiteness of a whitened oral surface. Suitably therefore the compositions of the invention may be for use in prolonging the effectiveness of a whitening agent on an oral surface. Suitably the compositions of the invention may be for use in combination with a whitening agent. Suitably the whitening agent is contacted with the oral surface to produce a whitened oral surface, and subsequently the composition of the invention is contacted with the whitened oral surface.

Suitably, a method of prolonging the effectiveness of a whitening agent on an oral surface is provided, the method comprising the steps of:
(i) Contacting the oral surface with a whitening agent for a sufficient time to produce a whitened oral surface;
(ii) Contacting the whitened oral surface with a composition of the invention for a sufficient period of time to prolong the whiteness of the whitened oral surface.

Suitably the composition comprises a phosphopeptide. Suitably the composition is according to the first or second aspects.

Suitably the composition of the invention protects the whitened oral surface from staining and thereby preserves and prolongs the whiteness of the oral surface such that the whitened oral surface lasts longer before becoming stained compared to whitened oral surfaces that are not contacted with a composition of the invention.

Remineralisation and Prevention of Demineralisation:

Some aspects of the present invention relate to use of compositions in remineralisation and/or preventing demineralisation.

Suitably a composition for use in remineralisation and/or preventing demineralisation may or may not also prevent or reduce staining of an oral surface. In one embodiment, the composition for use in remineralisation and/or preventing demineralisation is also for use in preventing or reducing staining of an oral surface.

Suitably the compositions for use in remineralisation and/or preventing demineralisation may be liquid or colloid compositions, suitably if the composition is a liquid it is an aqueous liquid.

Suitably the compositions for remineralisation and/or preventing demineralisation may be a mouthwash, an oral spray or a toothpaste. Suitably such liquid or colloidal compositions have formulations as described elsewhere herein.

Suitably the term remineralisation as used herein means that that mineral deposits are made onto the oral surface. Suitably prevention of demineralisation as used herein means that the oral surface is protected from loss of minerals.

Suitably the compositions remineralise or prevent demineralisation of an oral surface by up to 100% compared with non-treated oral surfaces. Suitably the compositions remineralise or prevent demineralisation of an oral surface by 50%, 60%, 70%, 80%, 90%, 100% compared to non-treated oral surfaces.

Suitably the level of remineralisation or prevention of demineralisation may be measured using quantitative light induced fluorescence (QLF), or by Vickers microindentation as described in ASTM E384 method (American Society for Testing and Materials). https://www.astm.org/Standards/E384.htm.

In embodiments where the composition is a liquid, suitably the composition remineralise oral surfaces by up to 90% compared to a non-treated oral surface, suitably by between 70-90% compared to non-treated oral surface.

In embodiments where the composition is a colloid, suitably the composition remineralise oral surfaces by up to 80% compared to a non-treated oral surface, suitably by between 50-80% compared to a non-treated oral surface.

Suitably the oral surface may be a natural surface or a synthetic surface. Suitably the oral surface is hard. Suitably the oral surface is mineralized as explained above. Suitably in one embodiment, the oral surface is teeth, suitably the surface of teeth, suitably the enamel of teeth. Suitably the oral surface may comprise a lesion. Suitably a lesion may be carious and/or erosive. Suitably the lesion may be pre-cavity or may be a cavity. Suitably, therefore, the compositions for use in remineralisation and/or preventing demineralisation may remineralise lesions in oral surfaces. Suitably the compositions for use in remineralisation and/or preventing demineralisation may remineralise caries or carious lesions in oral surfaces. Suitably, a dental condition involving dental demineralisation may comprise lesions, suitably lesions in oral surfaces. Suitably, treatment or prevention of dental caries or a dental condition involving dental demineralisation may comprise remineralizing lesions in oral surfaces. Suitably, a dental condition involving dental demineralisation may comprise caries or carious lesions suitably in oral surfaces. Suitably, treatment or prevention of dental caries or a dental condition involving dental demineralisation may comprise remineralizing caries or carious lesions in oral surfaces.

In some embodiments, the compositions are for use in remineralisation and/or preventing demineralisation of enamel, suitably of surface enamel or of subsurface enamel. In some embodiments the compositions are for use in remineralisation and/or preventing demineralisation of subsurface enamel. Suitably, in some embodiments the compositions act by remineralizing the enamel, suitably remineralizing from the subsurface to the surface of the enamel. Suitably, in some embodiments the compositions for use in remineralisation and/or preventing demineralisation are applied to the subsurface enamel. Suitably, in some such embodiments the compositions are applied in combination with a source of fluoride.

Suitably the compositions for use in remineralisation and/or preventing demineralisation remineralise lesions in teeth. Suitably the compositions remineralise lesions in teeth by up to 100% compared with non-treated teeth. Suitably the compositions remineralise lesions in teeth by 50%, 60%, 70%, 80%, 90% compared to non-treated teeth.

In embodiments where the composition is a liquid, suitably the composition remineralizes carious lesions in teeth by up to about 90% compared to a non-treated teeth, suitably by between 70-90% compared to non-treated oral surface.

In embodiments where the composition is a colloid, suitably the composition remineralizes carious lesions in teeth by up to about 80% compared to a non-treated teeth, suitably by between 50-80% compared to a non-treated oral surface.

Suitably the compositions for use in remineralisation and/or preventing demineralisation reharden lesions in teeth. Suitably the compositions reharden lesions in teeth in a rapid and progressive way, approaching complete remineralisation of the enamel. Suitably by up to about 100% compared with non-treated teeth. Suitably the compositions reharden lesions in teeth by 50%, 60%, 70%, 80%, 90% or even up to 100% compared to non-treated teeth.

In embodiments where the composition is a colloid, suitably the composition rehardens carious lesions in teeth in a rapid and progressive way approaching complete remineralisation of the lesion. Suitably by up to about 80% compared to a non-treated teeth, suitably by between 50-80% compared to a non-treated oral surface.

Suitably the remineralisation process may be measured by mineral density. Suitably the compositions of the invention increase the mineral density of an oral surface, suitably of enamel. Suitably, therefore the compositions are for use in remineralisation and/or preventing demineralisation and/or prevention or treatment of dental caries by increasing the mineral density of an oral surface, such as enamel. Suitably, therefore the compositions are for use in remineralisation and/or preventing demineralisation and/or prevention or treatment of dental caries by increasing the mineral density of surface or subsurface enamel. Suitably the compositions of the invention increase the mineral density of an oral surface by between 70-100% compared to non-treated oral surface. Suitably mineral density of an oral surface such as enamel may be measured by Qualitative Light Fluorescence (QLF) (Journal of Dentistry, 2013, 41(2): 127-132, Alammari MR1, Smith P W, de Josselin de Jong E, Higham S M).

Suitably the compositions for use in remineralisation and/or preventing demineralisation, or for treatment or prevention of dental caries or a dental condition involving dental demineralisation may be applied to an oral surface at least twice per day, suitably three times per day. Suitably, when the oral surface is within the mouth of a subject, the compositions for use in remineralisation and/or preventing demineralisation, treatment or prevention of dental caries or a dental condition involving dental demineralisation, may be for administration to the subject at least twice per day, suitably three times per day, suitably to the mouth of the subject. Suitably, when the oral surface is within the mouth of a subject, the compositions for use in remineralisation and/or preventing demineralisation, treatment or prevention of dental caries or a dental condition involving dental demineralisation, may be for administration to the subject at three monthly intervals.

Suitably the composition is applied to an oral surface, or is for administration to a mouth of a subject, at least twice per day for at least 5 days, at least 6 days, at least 7 days, at least 8 days, at least 9 days, at least 10 days, at least 11 days, at least 12 days, at least 13 days, at least 14 days, at least 15 days.

Suitably, the higher the number of consecutive days on which the composition is applied or administered, the higher the effect of remineralisation and/or prevention of demineralisation. Suitably therefore, frequent application of the formulations of the invention leads to progressive remineralisation of an oral surface.

Suitably, application of a composition of the invention to an oral surface every day for at least 40 days remineralizes a lesion back to sound enamel.

In embodiments where the composition is a liquid, suitably the composition is applied to an oral surface, or is for administration to a mouth of a subject as frequently as possible. Suitably, the more frequently applied and over an extended time period, the better the remineralizing effect. Suitably the composition is applied to an oral surface, or is for administration to a mouth of a subject at least twice per day for at least 7 days. Suitably this is sufficient to provide an improvement in remineralisation and/or prevention of demineralisation over non-treated oral surfaces. Suitably the improvement may be up to about a 45% increase in remineralisation of an oral surface compared to non-treated oral surfaces. Suitably, progression of the erosive lesion remineralisation tends towards complete remineralisation in a few days.

In embodiments where the composition is a colloid, suitably the composition is applied to an oral surface, or is for administration to a mouth of a subject as frequently as possible. Suitably, the more frequently applied and over an extended time period, the better the remineralizing effect. Suitably the composition is applied to an oral surface, or is for administration to a mouth of a subject at least twice per day. for at least 5 days. Suitably this is sufficient to provide an improvement in remineralisation and/or prevention of demineralisation over non-treated oral surfaces. Suitably the improvement may be up to about a 45% increase in remineralisation of an oral surface compared to non-treated oral surfaces. Suitably, progression of the erosive lesion remineralisation tends towards complete remineralisation in a few days.

Enhancing the Buffering Capacity of Saliva

Further aspects of the invention relate to a composition comprising a phosphopeptide for use in remineralisation and/or preventing demineralisation of an oral surface, or for the treatment or prevention of dental caries and/or erosive lesions, by increasing the buffering capacity of saliva. In one embodiment, there is provided a composition comprising a phosphopeptide, and optionally monofluorophosphate, for use in remineralisation and/or prevention of demineralisation of an oral surface by increasing the buffering capacity of saliva. In one embodiment, there is provided a composition comprising a phosphopeptide, and optionally monofluorophosphate, for use in the treatment or prevention of dental caries or a dental condition involving dental demineralisation by increasing the buffering capacity of saliva. In some embodiments, the composition does not comprise additional phosphate or an additional source of phosphate as described hereinabove.

By ‘buffering capacity’ it is meant the ability of saliva to buffer against changes in pH caused by acidic foods and bacterial action. Buffering capacity may be defined as the resistance to change of pH of a solution containing a buffering agent. Buffering capacity is the concentration of protons required to shift the pH of a solution 1 unit either side of the pKa H+/(L saliva*pH unit).

The increase in buffering capacity of saliva provided by the compositions of the invention contributes to preventing demineralisation and to treatment and prevention of dental caries, by enhancing acid neutralisation, preventing acid erosion and by enhancing enamel remineralisation.

In some embodiments, the composition may act to remineralise an oral surface by stabilising the buffering capacity of saliva. Suitably, the composition may act as a buffer reservoir.

In a further aspect of the invention there is provided the use of a composition comprising a phosphopeptide for increasing the buffering capacity of saliva. In a further aspect of the invention there is provided the use of a composition comprising a phosphopeptide as a buffer reservoir for saliva. In a further aspect of the invention there is provided the use of a composition comprising a phosphopeptide as an artificial saliva. Suitably the composition is a composition as described herein. Suitably the use is in a subject in need thereof, or for use in the treatment of a subject in need thereof. Suitably the buffering capacity is increased in a subject in need thereof, suitably in the saliva of a subject thereof.

Suitably the composition which increases or enhances the buffering capacity may be a composition of the invention as described elsewhere herein. In some embodiments, the composition which increases or enhances the buffering capacity may be a composition selected from MOK, MOL or MON as described herein.

Suitably, the composition is applied to an oral surface, or is for administration to a mouth of a subject, as frequently as possible to increase the buffering capacity of saliva. Suitably, the more frequently the composition is applied or administered, and the longer the duration of application, the better the remineralizing effect. Suitably the composition is applied to an oral surface, or is for administration to a mouth of a subject, at least twice per day for at least 7 days. Suitably the composition may be applied to the oral surface or administered as a spray. Suitably this is sufficient to provide an improvement in remineralisation over non-treated oral surfaces. Suitably the improvement may be up to about a 45% increase in remineralisation of an oral surface compared to non-treated oral surfaces. Suitably, progression of the remineralisation tends towards complete remineralisation in a few days.

Without being bound by theory, the inventors have discovered that a composition comprising a phosphopeptide with a high bicarbonate concentration acts as a reservoir of buffer when administered to the mouth. Use of such a composition of the present invention coats the surfaces of the oral cavity and enhances both day time and especially night time saliva buffering. Enhanced saliva buffering during both day and night time results in reduced demineralisation of the dental enamel and reduced risk of dental caries. The buffering capacity of saliva is most reduced at night time, therefore administering a composition of the present invention prior to sleep has the advantage of reducing acid erosion and caries formation when the buffering capacity of oral saliva is low.

The compositions of the invention act as a reservoir that further buffers the saliva against acidic pH which is especially effective when administered as a spray. Use of the composition of the invention coats the surfaces of the oral cavity and enhances both day time and night time saliva buffering. The inventors have shown that a pH titration of a composition of the invention such as Toothboost increases the oral buffering capacity to 17.0 mM H+/(L*pH unit). This equates to a 2.3× increase in the buffering capacity compared to human saliva alone. The application of a composition of the invention to the oral cavity by spraying action, provides an evenly dispersed and stable reservoir of buffer in the mouth, which enhances the remineralisation effect.

Suitably, therefore, in some embodiments, the composition of the invention may be administered prior to sleep. In some embodiments, the composition of the invention may be administered in the evening or at night time. In one embodiment, the composition of the invention may be administered prior to sleep to increase the evening or night time buffering capacity of saliva. In one embodiment, the composition of the invention may be administered prior to sleep to remineralise, and/or prevent or treat demineralisation or conditions involving dental demineralisation such as dental caries by increasing the buffering capacity of saliva. In one embodiment, the composition may be administered prior to sleep to provide an evening or night time buffering reservoir. In some embodiments, the evening or night time buffering reservoir is increased in the subject. In one embodiment, the composition of the invention may be administered prior to sleep to remineralise, and/or prevent or treat demineralisation or condition involving dental demineralisation such as dental caries by increasing the evening or night time buffering capacity of saliva.

Suitably the composition of the invention may be administered to the mouth of a subject, and/or may be applied to an oral surface. Suitably the composition may be administered or applied as a spray or as a paste. In one embodiment, the composition is administered a spray which is effective for coating the oral surfaces as explained above. Suitably the composition may be MOL or MON.

In one embodiment, the composition of the invention may be administered prior to sleep to remineralise, and/or prevent or treat demineralisation or conditions involving dental demineralisation such as dental caries by increasing the buffering capacity of saliva, wherein the composition is administered as a spray.

Packaging:

In an aspect of the present invention there is provided packaging comprising a composition of the invention, suitably an oral care composition according to the fifth aspect, or a composition for use according any of the aspects of the invention.

Suitably the packaging contains the composition of the invention. Suitably the packaging may also be operable to deliver the composition of the invention, suitably to an oral surface. Suitably the packaging may comprise a container to contain the composition. Suitably the packaging may further comprise an applicator or an actuator.

Suitably the packaging may comprise an actuator. Sutiably upon actuation of the actuator, the composition is expelled from the packaging, suitably from within the container of the packaging. Suitably, the actuator is operable to deliver a metered dose of the composition when actuated. Suitably a metered dose is a suitable amount of the composition to achieve the desired effect. Suitably a metered dose is an amount suitable for a mouth of a subject, for example between 0.1 ml up to 5 ml.

Suitable actuators may include a spray nozzle. Suitably, upon actuation of the spray nozzle, droplets of the composition are expelled from the packaging.

Sutiably the packaging may further comprises a propellant, suitably the propellant aids expelling the composition from the packaging. Suitably, in such an embodiment, the spray nozzle is an aerosolization spray nozzle.

In one embodiment, the packaging comprises a spray nozzle. Suitably, in such an embodiment, the packaging may be termed a ‘spray pack’. Suitably, in such an embodiment, the composition is an oral spray.

Embodiments of the present invention will now be described, by way of non-limiting example, with reference to the accompanying drawings.

### BRIEF DESCRIPTION OF THE FIGURES

FIG. 1: Vickers Microindentation results for remineralisation of erosive lesions in bovine enamel after treatment with: ToothBoost formulation MOK, ToothBoost formulation MOL, 500 ppm F as MFP or deionised water. N=8 bovine enamel, treated for seven days.

FIG. 2: The remineralisation of caries lesions after treatment with either deionised water, a chewable mint tablet solution or ToothBoost formulation MOL every day for 41 days, measured by QLF.

FIG. 3: Vickers Microindentation to measure rehardening of erosive lesions in human enamel after treatment with: fluoride free Toothboost MOL formulation, 500 ppm F as MFP or deionised water. N=6 human enamel, treated for five days.

FIG. 4: The titration of Toothboost with sodium hydroxide showing the end point, Pka and buffering capacity at ½ EP +/−1 pH unit.

FIG. 5: Change in colour, ΔE, for enamel discs in a stain prevention cycling study after treatment with one of five test formulations. N=10 enamel specimens per treatment.

FIG. 6: Change in colour, ΔE, for bovine enamel in a stain prevention cycling study after treatment with one of four test formulations. N=10 enamel specimens per treatment.

FIG. 7: Overall the scheme of results of Example 6.

FIG. 8: Change in colour, ΔE, for retainer incubated in ToothBoost or deionised water at 37° C. for 117 days. N=10 Orthodontic polymer discs.

FIG. 9: Vickers Microindentation to measure the rehardening of acid etched human enamel after treatment with: BoostPaste® formulation MON, 1450 ppmF MFP and deionised water. N=8 human enamel specimens per treatment.

FIG. 10: Vickers Microindentation to measure the rehardening of citric acid erosive lesions in human enamel after treatment with: fluoride free BoostPaste formulation MON-6, 1450 ppmF MFP and deionised water over 5 days (from left to right=day 1 to day 5). N=10 human enamel specimens per treatment.

FIG. 11: A) total fluoride; B) free fluoride; C) free calcium; D) pH; E) % Transmittance at 400 nm over 10 months of storage of ToothBoost formulation MOL (from left to right=1 month, 2 months, 3 months, 8 months, 10 months).

FIG. 12: The increase in mineral content of erosive lesions after treatment with Tooth boost of the invention, or 1450 ppm fluoride (NaF), five times a day for five days

FIG. 13a: The remineralisation of subsurface caries lesions after treatment with Toothboost MOL-3 formulation once a day over a 50 day treatment period.

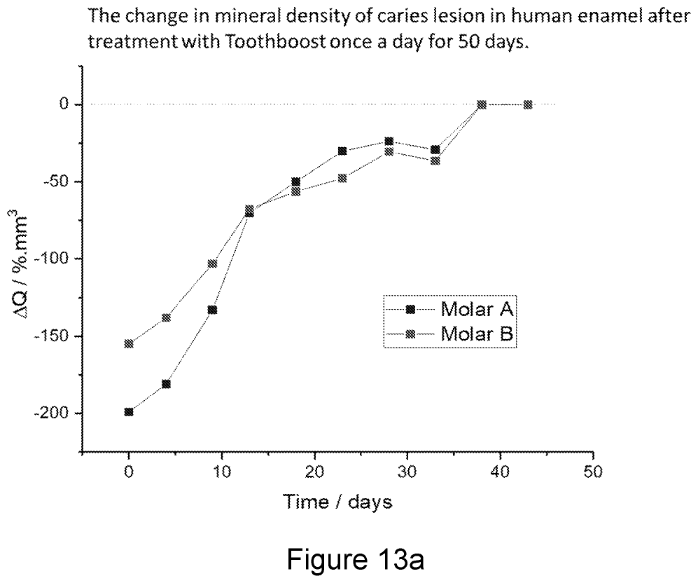

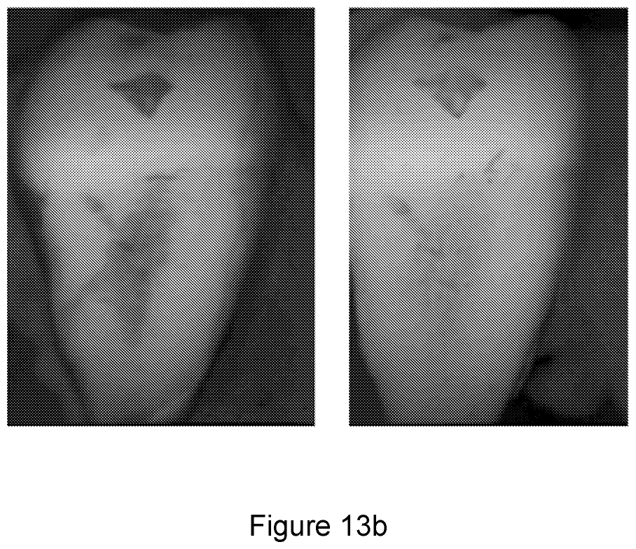

FIG. 13b: An example showing the measurement of enamel remineralisation
for an artificial caries lesion. The measurement of diamond shaped lesion
by QLF gives detailed measurements in mineral density

### DETAILED DESCRIPTION OF EMBODIMENTS OF THE INVENTION AND EXAMPLES

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term ‘about’ as used herein may refer to +/−20%, +/−15%, or +/−10% of the value recited, suitably +/−10% of the value recited.

The term “ToothBoost” is used herein at some points in the examples to describe liquid compositions according to the present invention such as ‘MOL’ as prepared in example 1.

The term “Boostpaste” is used herein at some points in the examples to describe paste compositions according to the present invention such as MON′ as prepared in example 2.

‘MOK’ refers to a previous comparative liquid formulation containing phosphate in the form of phosphate buffers, the formulation of which is as follows:

MOK batches

| Intermediate A | |
|---|---|
| Ingredient | % w/w |
| Sodium Methyl Paraben | 0.2 |
| Phenoxyethanol | 2.6 |
| Saccharine | 1.0 |
| Tego Betain | 7.7 |
| Deionised water | 81.5 |
| Flavour oil | 7.0 |

| Finished Product | |
|---|---|
| Ingredient | % w/w |
| OPN-10 | 3.0 |
| Trisodium phosphate 0.1M solution | 16.0 |
| Disodium hydrogen phosphate 0.1M solution | 16.0 |
| Sodium bicarbonate 1M solution | 15.0 |
| Deionised water | 30.0 |
| calcium chloride 1M solution | 2.9 |
| MFP | 0.4 |
| xylitol | 5.0 |
| Intermediate A | 7.7 |
| make up to volume after final pH adjustment | 4.0 |

To make the Intermediate A flavour system:

1. Mix Phenoxyethanol with flavour oil.

2. Disperse Tego Betain into the solution by mixing.

3. Mix saccharin, methyl paraben and the deionised water until a clear solution.

4. Add this quickly to the tego betain suspension and stir. After about an hour it is a clear straw colour solution.

To make the finished product:

1. Add 2/3 of the total volume of deionised water to 3.0% w/w OPN-10 and rapidly mix for two hours until the solution clears.

2. Add 1M calcium chloride at a rate of 0.3 ml/min with rapid stirring.

3. Add tri-sodium phosphate and di-sodium hydrogen phosphate to the OPN-10 solution at a rate of 0.4/min.

4. Add monofluorophosphate solution made from dissolving MFP into ⅓ of the total volume of deionised water. Add back to the OPN solution at a rate of 0.2 ml/min. This will result in a very slightly cloudy pale yellow/white solution.

5. Add the sodium bicarbonate solution at a rate of 0.3 ml/min, keeping pH at 7.5±0.3 with 1M HCl/1M NaOH.

6. Add intermediate A to the OPN solution at a rate of 0.2 ml/min with rapid stirring. This will result in a clear solution.

7. Mix in 5.0% w/w xylitol.

8. Adjust the pH of the solution to 7.5.

9. Make up to 100.0 g with deionised water.

10. After 24H Filter through a 0.22 um sterile filter.

If using a single transfer tube for the reagents, it must be washed through before each reagent is added.

### Example 1—Novel Liquid Composition Comprising Phosphoproteins (Phosphate Free)—“MOL”

MOL batches Fluoride free.

| Ingredient | % w/w |
|---|---|
| Intermediate A | |
| Sodium Methyl Paraben | 0.2 |
| Phenoxyethanol | 2.6 |
| Saccharine | 1.0 |
| Tego Betain | 7.6 |
| Deionised water | 81.5 |
| Flavour oil | 6.9 |
| Finished Product | |
| OPN-10 | 3.0 |
| Sodium bicarbonate 1M solution | 15.0 |
| Deionised water. | 54.0 |
| Calcium chloride 1M solution | 2.9 |
| Xylitol | 5.0 |
| HCl 1M solution | 11.0 |
| Intermediate A | 7.7 |
| Make up to final volume with water | 1.4 |
| after final pH adjustment | |

MOL batches with fluoride

| Ingredient | % w/w |
|---|---|
| Intermediate A | |
| Sodium Methyl Paraben | 0.2 |
| Phenoxyethanol | 2.6 |
| Saccharine | 1.0 |
| Tego Betain | 7.6 |
| Deionised water | 81.5 |
| Flavour oil | 6.9 |
| Finished Product | |
| OPN-10 | 3.0 |
| Sodium bicarbonate 1M solution | 15.0 |
| Deionised water. | 54.0 |
| Monoflurophosphate | 0.4 |
| Calcium chloride 1M solution | 2.9 |
| Xylitol | 5.0 |
| HCl 1M solution | 11.0 |
| Intermediate A | 7.7 |
| Make up to final volume with water | 1.0 |
| after final pH adjustment | |

Manufacture of flavour system Intermediate A.
1. Mix Phenoxyethanol with flavour oil.
2. When a clear solution, disperse Tego Betain into the solution and mix until fully dispersed.
3. Mix saccharin and methyl paraben to deionised water and mix until dissolved.
4. Add the saccharine mix, quickly, to the tego suspension and stir. After about an hour it is a clear straw colour solution.

Manufacture of finished product (100 g).
1. Add 49 g of deionised water to a beaker and add 3.0 g of OPN and stir for two hours.
2. Add calcium solution.
3. Mix MFP with 5 ml of deionised water and add to bicarbonate solution and add xylitol and stir until dissolved.
4. Add sodium bicarbonate.
5. Add Intermediate A.
6. Add HCl solution.
7. Check and adjust pH after 24 hours.
8. Filter through a 0.2 μm sterile filter.

A phosphate free and fluoride free formulation of MOL can be made in the same way as detailed above with the same components except monofluorophosphate (MFP) is not included, see above Table.

### Example 2—Novel Paste Composition Comprising Phosphoproteins (Phosphate Free)—“MON”

MON batches

| Intermediate A | % w/w |
|---|---|
| Sodium Methyl Paraben | 0.2 |
| Phenoxyethanol | 2.6 |
| Saccharine | 1.0 |
| Tego Betain | 7.6 |
| Deionised water | 80.7 |
| Flavour oil | 7.8 |

| Finished product - with fluoride | |
|---|---|
| Ingredient | % w/w |
| OPN-10 | 3.0 |
| Sodium bicarbonate | 2.0 |
| Zeofree 153 | 12.0 |
| Abrasive silica | 6.0 |
| Deionised water. | 22.9 |
| HCl 1M | 34.0 |
| Calcium chloride solid | 0.5 |
| Monoflurophosphate | 1.1 |
| Manitol | 9.0 |
| Xanthan | 0.8 |
| Intermediate A | 7.7 |
| make up to with water after | 1.0 |
| final pH adjustment | |

| Finished product - fluoride free | |
|---|---|
| Ingredient | % w/w |
| OPN-10 | 3.0 |
| Sodium bicarbonate | 2.0 |
| Zeofree 153 | 12.0 |
| Abrasive silica | 6.0 |
| Deionised water. | 22.9 |
| HCl 1M | 34.0 |
| Calcium chloride solid | 0.5 |
| Manitol | 9.0 |
| Xanthan | 0.8 |
| Intermediate A | 7.7 |
| make up to with water after | 2.1 |
| final pH adjustment | |

Manufacture of flavour system Intermediate A.
1. Mix Phenoxyethanol with flavour oil.
2. When a clear solution, disperse Tego Betain into the solution and mix until fully dispersed.
3. Mix saccharin and methyl paraben to deionised water and mix until dissolved.
4. Add the saccharine mix, quickly, to the tego suspension and stir. After about an hour it is a clear straw colour solution.

Manufacture of finished product (100 g).
1. Add deionised water and 1M HCl and OPN and stir for 30 min.
2. Add sodium bicarbonate, calcium, Zeofree silica and Abrasive silica, MFP and Manitol.
3. Mix with a high sheer mixer until fully dispersed.
4. Add Intermediate A.
5. Mix in Xanthan.
6. Check pH by taking 2 g of the paste a make a 25% suspension. Adjust to 7.0, if required.

A phosphate free and fluoride free formulation of MON can be made in the same way as detailed above with the same components except monofluorophosphate (MFP) is not included.

### Example 3—Remineralisation of Erosive Lesions in Bovine Enamel after Treatment with Formulation MOK Compared with Formulation MOL

Introduction. The remineralisation potential of two formulations MOK (MOK-5), MOL (MOL-1), (the numbers indicate the number of the batch), 500 ppm F solution (as MFP) and deionised water were tested in a standard Microindentation erosive lesion rehardening model as described in ASTM E384 method (American Society for Testing and Materials). https://www.astm.org/Standards/E384.htm. The objective of the study was to ascertain if formulation development for ToothBoost retained remineralizing efficacy. Four treatment groups were placed in the study: 1. MOK (legacy formulation), 2. Phosphate free ToothBoost MOL (development formulation), 3. 500 ppm fluoride solution made from MFP, 4. Deionised water. N=8 enamel specimens per treatment group.

Enamel blocks are prepared from sound disease free human molars or bovine incisors and mounted in 25 mm diameter resin discs. After curing the underside of the disc is flattened using p400 grit paper. The enamel side is ground with p800 paper to expose the enamel and then serially polished using 1200 and then 2500 grit paper and finally 1 um diamond polish.

The discs are then rinsed under deionised water and sonicated for five minutes. Initial microhardness of the sound enamel is measured by microindentation to determine that the enamel is sufficiently mineralized for the purpose of the study and to record the microhardness of the sound enamel. The acceptance criteria is >350VHN (human) >300VHN (bovine).

Method. The sound enamel blocks are placed into a water bath, enamel side up, and 1% citric acid solution at pH3.75 is added so that there is an excess of solution. The enamel specimens are then incubated at 37° C. for ten minutes to form erosive lesions. The citric acid solution is discarded and the enamel rinsed in copious volumes of de-ionised water for ten minutes. The microhardness of the de-mineralized enamel is then measured and the enamel discs organised so that each group have a similar range of microhardness values.

The citric acid eroded bovine enamel specimens were then treated twice a day for seven days. The treatments were applied using a single actuation of a spray pack delivering approximately 0.1 ml onto the surface of the enamel. The enamel was then incubated in artificial saliva (containing 1500 units/L of phosphatase) at 37° C. between treatments. At the end of day seven, the enamel specimens were rinsed in deionised water, dried and the micro hardness of the enamel was measured by Vickers microindentation to determine the extent of the re-mineralization of the enamel lesions. Each hardness value was determined from the average of ten individual measurements made centrally on each enamel specimen using 1.9N force over an indent time of 20 seconds. At the start of each set of measurements a calibration check is performed using a standard metal block to ensure the reproducibility of microhardness is within 3% of the test block microhardness.

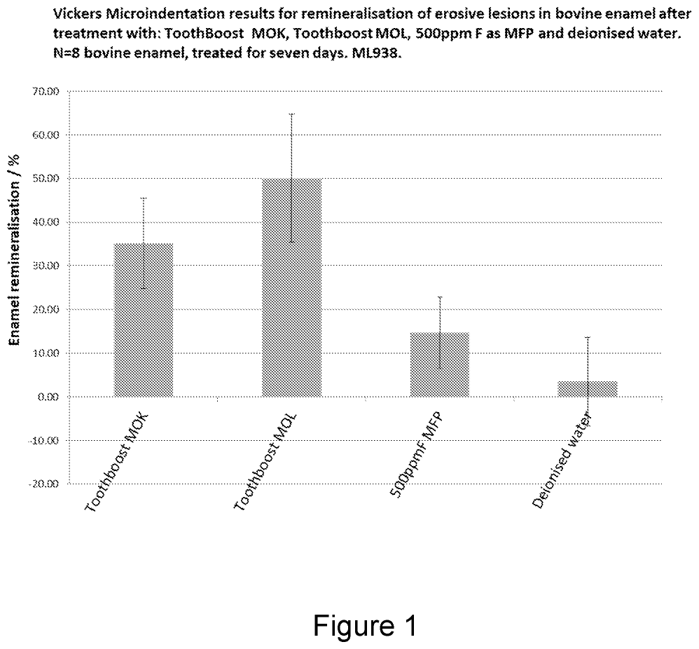

Results. The result of the microindentation study are shown in FIG. 1. They show that the 500 ppm fluoride solution is statistically (probability <0.05) better than deionised water. Both of the OPN formulations MOK and MOL are statistically better than 500 ppm fluoride. The two OPN formulations, MOK and MOL are statistically the same. Overall the trend in remineralisation followed the scheme: OPN (MOL)=OPN (MOK)>500 ppm fluoride solution >Deionised water

### Example 4—the Measurement of the Remineralisation of Lactic Acid Caries Lesion in Enamel after Treatment with MOL. Measured by Quantitative Light Induced Fluorescence (Qlf)

Introduction. A study to measure the remineralisation of caries lesions formed in bovine enamel using a ten-day lactic acid gel system. After formation of the lesions the specimens were treated with one of three test formulations twice a day. The enamel were rinsed in deionised water between treatments and incubated in artificial saliva. Periodically the enamel specimens were removed from the artificial saliva, rinsed and dried and the caries lesion examined using quantitative light induced fluorescence (QLF) to determine the degree of remineralisation. The QLF software time-laps animated sequence was rendered into a video showing the continuous process of the remineralisation over the treatment period. In addition, the QLF results gave quantitative values for the remineralisation. Each formulation was tested using n=10 enamel specimens per treatment. The treatments were, 1. Deionised water, 2. Commercial Chewable mint tablet, 3. ToothBoost MOL-4.

Method. Caries lesions were formed in the enamel specimens by incubation in a lactic acid gel system for ten days at 37° C. The enamel was then treated using the following cycling procedure:
N=10 incisors were randomly selected for each of the three treatment groups and had a dedicated container for the treatment steps.
The enamel specimens were placed into artificial saliva for 1 hour before the start of the study.
The test formulations were decanted into a 20 ml spray pack, except for the Commercial chewable mint tablets, which were made into a solution and delivered by pipette.
The enamel was sprayed with a single actuation of the spray pack and incubated for five minutes.
The enamel was then rinsed with deionised water using a wash bottle.
The enamel was then placed into artificial saliva with phosphatase.
The process was repeated twice a day for up to 40 days.
Periodically, the enamel was rinsed, dried and placed into the lnspektor diagnostic QLF equipment to be measured.

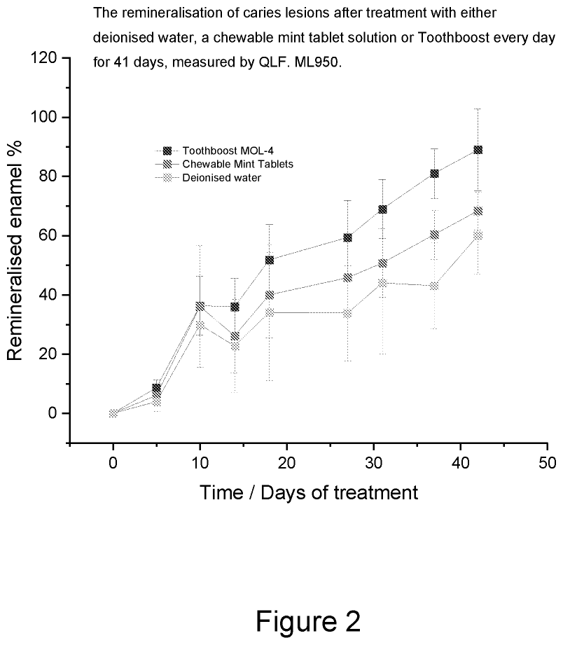

Results. The results are shown in FIG. 2 and the QLF measurements show that after day 25 measurement ToothBoost treated caries lesions remineralized statistically greater (p<0.05) that either Commercial chewable mint tablets or deionised water. The commercial chewable mint tablet is statistically the same as deionised water. After 30 days ToothBoost continues to remineralise caries lesions better than either Commercial chewable mint tablets or deionised water and Commercial chewable mint tablets is statistically better than deionised water. This remains the case to the end of the study period where ToothBoost has remineralized the caries lesions by 88%.

### Example 5—Measurement of the Remineralisation of Citric Acid Erosive Lesions in Enamel after Treatment with Fluoride Free Toothboost MOL Formulation

Introduction. The remineralisation potential of three formulations: deionised water, 500 ppm fluoride solution from MFP and fluoride free Toothboost MOL-15 formulation were tested in a standard microindentation erosive lesion rehardening model, as described above in example 3. The objective of this study was to ascertain if fluoride free Toothboost MOL-15 formulation has the potential to remineralise erosive lesions without the presence of fluoride.

Method. Five times a day for five days human enamel specimens with citric acid erosive lesions were sprayed with 0.15 g of the test formulations and incubated in artificial saliva (containing 1500 units/L of phosphatase) at 37° C. between treatments. The microhardness of the enamel specimens was remeasured after five days and the degree of re-mineralization calculated using the boundary conditions of 100% mineralized for the hardness of the initial enamel before the formation of the lesions and 0% mineralization for the hardness of the enamel after the formation of the lesion.

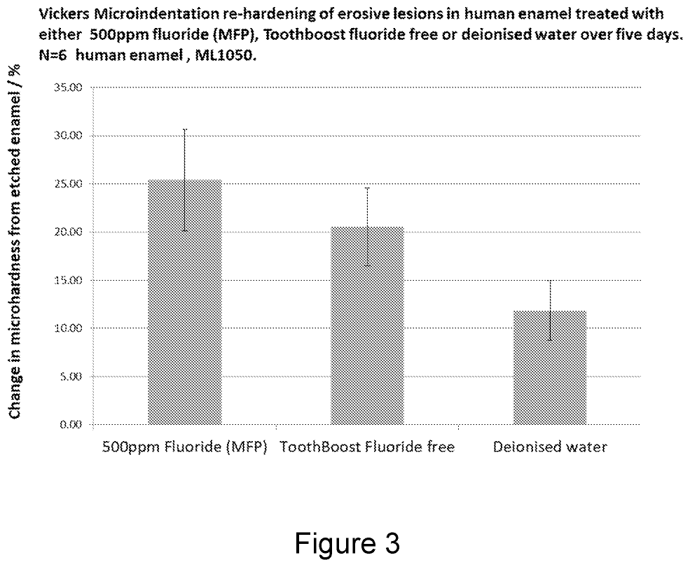

Results. The statistical analysis of the microindentation results shows that fluoride free Toothboost and 500 ppm fluoride solution provide significant remineralisation of the erosive enamel lesions after 5 days of treatment. After five days of treatment, the calculated remineralisation from the fluoride free Toothboost treated enamel was over 20% and from the fluoride solution was over 25%. See FIG. 3.

### Example 6— Measurement of the Buffering Capacity of Toothboost MOL Formulation

Introduction: The average buffering capacity of saliva from healthy adult volunteers during the day is measured as 7.25 mM H+/(L saliva*pH unit) (Archives of Oral Biology. Volume 45, Issue 1, January 2000, Pages 1-12). During sleep, the buffering capacity from bicarbonate buffer significantly drops and there have been extensive studies to show a correlation between saliva buffering capacity and prevalence of caries. Some have reported that a buffering capacity of 0.43 mM H+/(L saliva*pH unit) corresponds to a rampant condition of caries.

Method: MOL Toothboost was titrated with 1M sodium hydroxide to end point. The Pka and buffering capacity at ½ end point +/−1 pH unit is then calculated as H+/(L*pH unit).

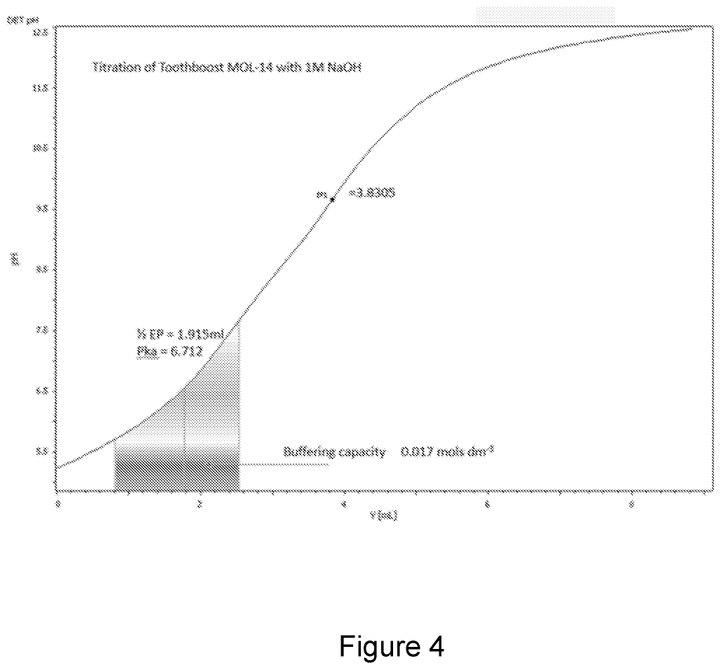

Results: The pH titration of Toothboost shows an increase in the oral buffering capacity to 17.0 mM H+/(L*pH unit). This equates to a 2.3× increase in the buffering capacity compared to human saliva alone. See FIG. 4 for a graph of the results. The treatment acts as a reservoir of Toothboost that further buffers the saliva against acidic pH when administered using the Toothboost misting technology.

### Example 7—Measurement of Stain Prevention Using Bovine Enamel

Introduction. A stain prevention study where bovine enamel specimens were treated with one of four mouthwash formulations and ToothBoost and then subjected to a cycling staining

procedure. The objective of the study was to determine which of the five formulations gave the best stain prevention as determined by a change in ΔE₇₆, the colour difference between the initial non-stained enamel and the enamel during the staining procedure. The treatments were ToothBoost MOL-6, Colgate Max White Expert, Listerine Advanced white, Corsodyl Original, deionised water.

Method. Carried out according to the method recited in ‘In vitro evaluation of a novel 6% hydrogen peroxide tooth whitening product’ by Andrew Joiner, Gopal Thakker et al. Journal of Dentistry (2004) 32, 19-25.

Bovine incisors are selected and 1 cm diameter cores taken and mounted in 2.5 cm diameter resin discs. The enamel is then polished flat using 800, 1200 and then 2500 grit paper and finally diamond polish. A baseline colour measurement is made of the initial non treated enamel using a colour meter. The minimum whiteness acceptance criteria is L*>70 measured from the initial L*a*b* colour values of the non-treated enamel.

N=10 bovine discs per treatment group were selected so that each treatment group had a similar range of L* whiteness values. In a dedicated water bath, the enamel was cycled between artificial saliva, tea stain and chlorhexidine and for a minimum of five cycles. The L*a*b* values were then measured using a Minolta Lab colour meter. For two more visits, the enamel was cycled for five cycles and the L*a*b* re-measured. From the L*a*b* values of initial, after cycle 5, cycle 10 and cycle 15, the ΔE₇₆ values were calculated where ΔE₇₆ (The difference between enamel colour before and after cycling) was determined from equation 1:

ΔE₇₆=\sqrt{(L1−L2)²++(a1−a2)²+(b1−b2)²} Eqn. 1

The cycling procedure is as follows:

| Cycle | Solution | Time | |
|---|---|---|---|
| 1 | Artificial saliva | 5 | mins |
| | Treatment | 2 | min |
| | Tea | 10 | min |
| | Chlorhexidine | 20 | min |
| 2 | Artificial saliva | 5 | mins |
| | Tea | 10 | min |
| | Chlorhexidine | 20 | min |
| 3 | Artificial saliva | 5 | mins |
| | Tea | 10 | min |
| | Chlorhexidine | 20 | min |
| 4 | Artificial saliva | 5 | mins |
| | Tea | 10 | min |
| | Chlorhexidine | 20 | min |
| 5 | Artificial saliva | 5 | mins |
| | Tea | 10 | min |
| | Chlorhexidine | 20 | min |

Artificial saliva (AS):

| | Mols dm⁻³ | |
|---|---|---|
| Magnesium Chloride | 0.2 | mM |
| Calcium chloride di-hydrate | 1.0 | mM |
| Potassium di-hydrogen orthophosphate | 4.0 | mM |
| HEPES (N-2Hydroxyethylpiperazine-N′- | 20 | mM |
| ethanethesulphonic acid) | | |
| Potassium chloride | 16.0 | mM |
| Ammonium chloride | 4.5 | mM |
| pH 7.0 with sodium hydroxide | | |

Chlorhexidine (CHX): Chlorhexidine 1% solution in deionised water pH adjusted to 5.9.
The stain solution: The tea stain is made from Tetley English Breakfast tea and is made by adding one tea bag into 0.1 L of freshly boiled water and left to infuse for five minutes.

The treatment is to place the enamel discs into an excess of the test formulation and time for 1 minute.

The treatment sequence is to place into artificial saliva, test treatment (at the first cycle only), chlorhexidine 0.2% and freshly brewed black tea. The groups are cycled between the three solutions without rinsing between treatments. After the fifth treatment cycle the enamel was rinsed with deionised water and allowed to dry before colour measurements were made. Fresh solutions were made at the start of each set of five cycles.

Colorimetry measurements are made on the initial enamel and then after treatments. Each colour measurement is determined from the average of four individual measurements made after rotating the enamel by 90°. The differences in colour measurements between the initial and treated enamel are then used to calculate the ΔE₇₈ values.

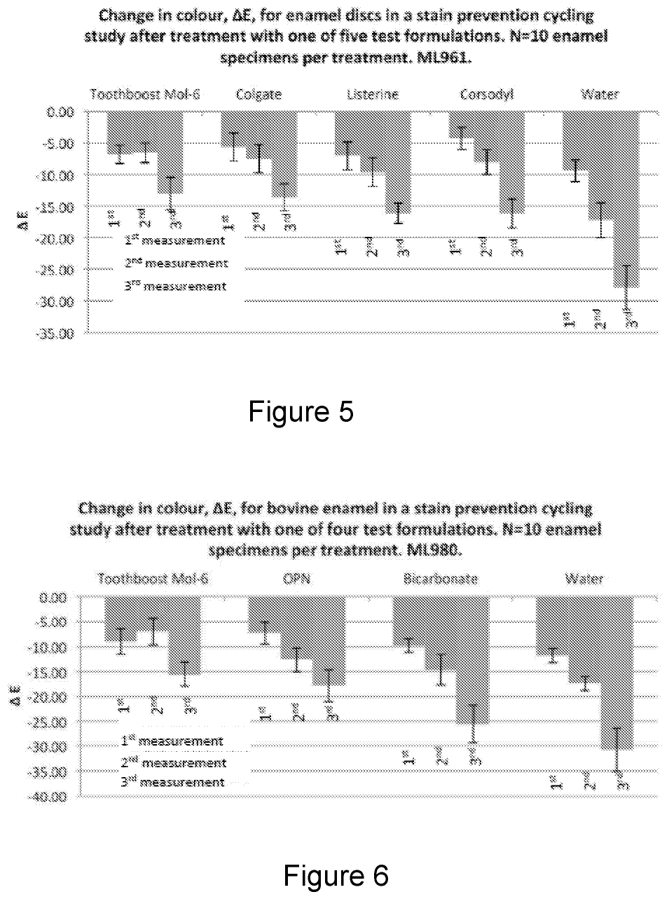

Results. The results from the study in in FIG. 5 and show that after the first measurement, Corsodyl® was statistically better than ToothBoost and statistically the same as Colgate. However, after the second and third measurements ToothBoost was statistically better than Corsodyl® and Listerine and statistically the same as Colgate. All the formulations were statistically better than deionised water at all time points.

### Example 8— Further Measurement of Stain Prevention Using Bovine Enamel

Introduction. A cycling staining procedure as described in example 6 was again used to measure the staining prevention when bovine enamel was subjected to a cycling staining procedure after being treated with one of four formulations: ToothBoost MOL-6, a 3% solution of OPN pH adjusted to 7.0, a 1.25% solution of sodium bicarbonate pH adjusted to 7.0 and deionised water. The aim of the study was to rank the formulations in terms of their stain prevention capability when bovine enamel was treated with a combination of tea stain and chlorhexidine.

Method. The enamel cores were treated by spraying the assigned treatment onto the enamel face using a single actuation of the ToothBoost spray pack and incubating for two minutes.

The enamel specimens were then cycled between a 1% chlorhexidine solution, a solution of strong tea and artificial saliva for approximately ten cycles per day. At the end of the day of cycling the enamel was rinsed and allowed to dry before the enamel colour measured. The cycling continued for a total of three days of cycling. From the L*a*b* measurements the ΔE₇₆ (change in perceivable colour) was calculated. ΔE₇₆₌₁ is the smallest perceivable colour change that can be observed.

The cycling procedure is as follows:

| Cycle | Solution | Time | |
|---|---|---|---|
| 1 | Artificial saliva | 5 | mins |
| | Treatment | 2 | min |
| | Tea | 10 | min |
| | Chlorhexidine | 20 | min |
| 2 | Artificial saliva | 5 | mins |
| | Tea | 10 | min |
| | Chlorhexidine | 20 | min |
| 3 | Artificial saliva | 5 | mins |
| | Tea | 10 | min |
| | Chlorhexidine | 20 | min |
| 4 | Artificial saliva | 5 | mins |
| | Tea | 10 | min |
| | Chlorhexidine | 20 | min |
| 5 | Artificial saliva | 5 | mins |
| | Tea | 10 | min |
| | Chlorhexidine | 20 | min |

The table shows the first five cycles of the ten cycle treatment. The cycling continued every day for three days.

After each set of cycling the enamel was rinsed with deionised water and allowed to dry before colour measurements were made. Fresh solutions were made at the start of each set of cycles.

Results. The change in colour of the enamel was calculated from the L*a*b* values where the ΔE₇₆ (The difference between enamel colour before and after cycling) was determined from equation 1:

ΔE₇₆=(L1−L2)²+(b1−b2)² Eqn. 1

After the first set of treatments, ToothBoost was statistically the same (i.e., gave the same degree of protection from staining) as OPN solution and sodium bicarbonate solution. OPN solution gave statistically more protection than sodium bicarbonate solution. All three were statistically better than deionised water.

After the second round of treatments, ToothBoost was statistically better than OPN and sodium bicarbonate solution. OPN and sodium bicarbonate were statistically the same. All three were statistically better than deionised water.

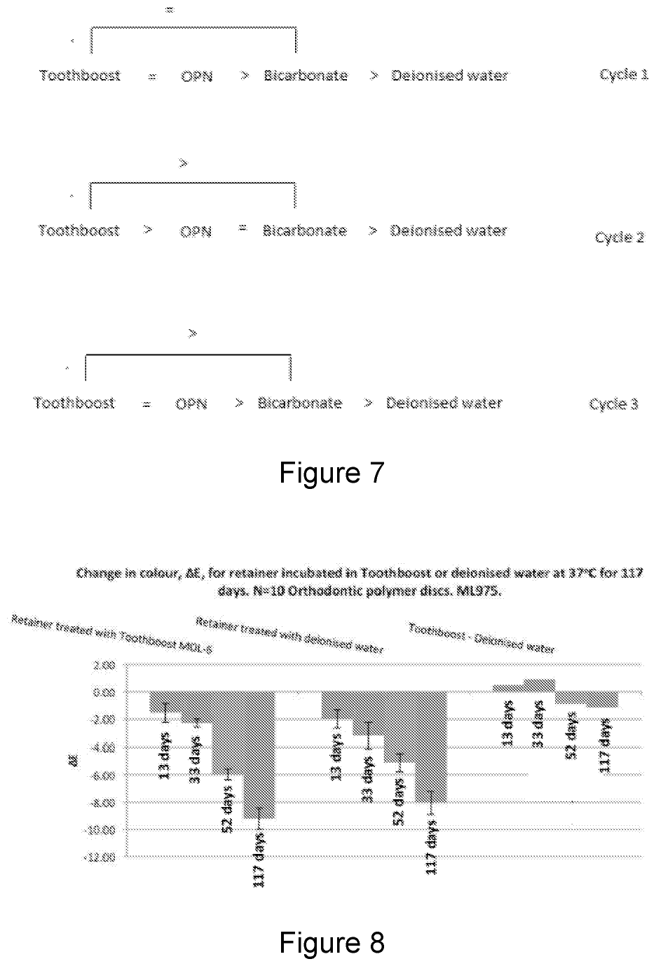

After the third round of treatments, ToothBoost and OPN solution were statistically the same and both OPN and ToothBoost were statistically better than sodium bicarbonate. All three were statistically better than deionised water. See FIG. 6 for a graph of the results. Overall the scheme as shown in FIG. 7 shows the pair wise statistical significance between the formulations at the three measurement points.

### Example 9—Orthodontic Retainer Polymer Staining Study

Introduction. The study is to show if treatment with ToothBoost whilst waring an orthodontic retainer caused staining to the retainer polymer. Sections of the retainer plastic were incubated in excess volumes of ToothBoost at 37° C. for up to 117 days. Periodically the retainers were removed from the ToothBoost solution, rinsed and dried and the colour of the retainer polymer was measured and compared to the initial colour of the retainer before treatment.

Method. 1 cm diameter retainer plastic cores were trepanned from Invisalign clear align orthodontic retainers and were used without further preparation. N=10 polymer discs were used for each treatment group which were 1. ToothBoost MOL-6, 2. Deionised water. The polymer discs were assigned to each of the two groups so that each group had a similar range of initial L* values measured using a Minolta spectrophotometer. In dedicated water baths, the group of 10 polymer discs were incubated in an excess of either ToothBoost or deionised water. After treatment at 13, 33, 52 and 117 days incubation the colour of the retainer polymer was measured and the L*a*b* values used to calculate the ΔE₇₆ (change in perceivable colour). (ΔE₇₆₌₁ is the smallest perceivable colour change that the human eye can detect).

Results: The results are summarised in FIG. 8 and show incubation of the retainers in pure deionised water causes the polymer to naturally discolour, to a small degree. Incubation in Toothboost also had the same degree of discolouration of about DE9.0 after 117 days.

Subtraction of the deionised water treated ΔE₇₆ values from the ToothBoost treated values indicate a ΔE₇₆ of about 1.0 over the 117 days treatment and is not statistically different from the colour of the initial polymer.

### Example 10—Measurement of the Remineralisation of Citric Acid Erosive Lesions in Enamel after Treatment with Boostpaste

Introduction. The remineralisation potential of three formulations: deionised water, 1450 ppm fluoride solution from MFP, and Boostpaste were tested in a standard microindentation erosive lesion rehardening model as described in ASTM E₃₈₄ method (American Society for Testing and Materials). https://www.astm.org/Standards/E384.htm and as described above in example 3. The objective of the study was to ascertain if Boostpaste, a new toothpaste format of ToothBoost, has the potential to remineralise erosive lesions. N=8 human enamel specimens were used per treatment group.

Method. Twice a day for five days human enamel specimens with citric acid erosive lesions were brushed with the test formulations and incubated in artificial saliva (containing 1500 units/L of phosphatase) at 37° C. between treatments. After day five the microhardness of the enamel specimens was remeasured and the degree of re-mineralization calculated using the boundary conditions of 100% mineralized for the hardness of the initial enamel before the formation of the lesions and 0% mineralization for the hardness of the enamel after the formation of the lesion.

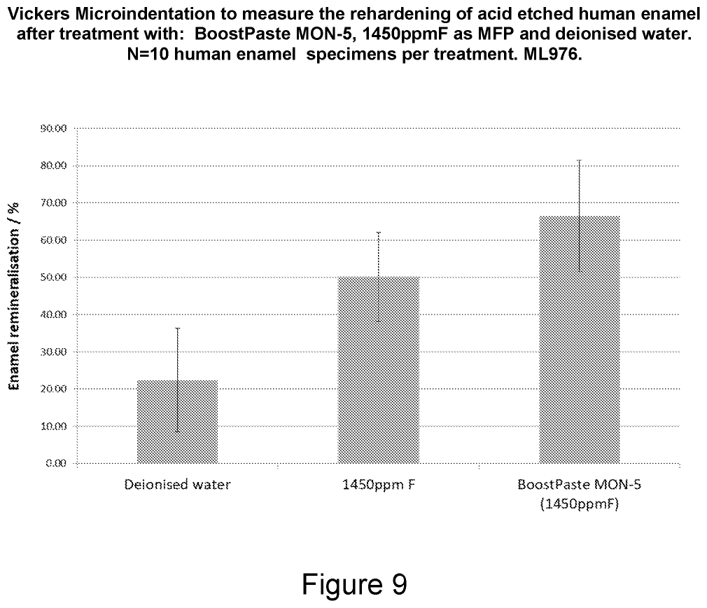

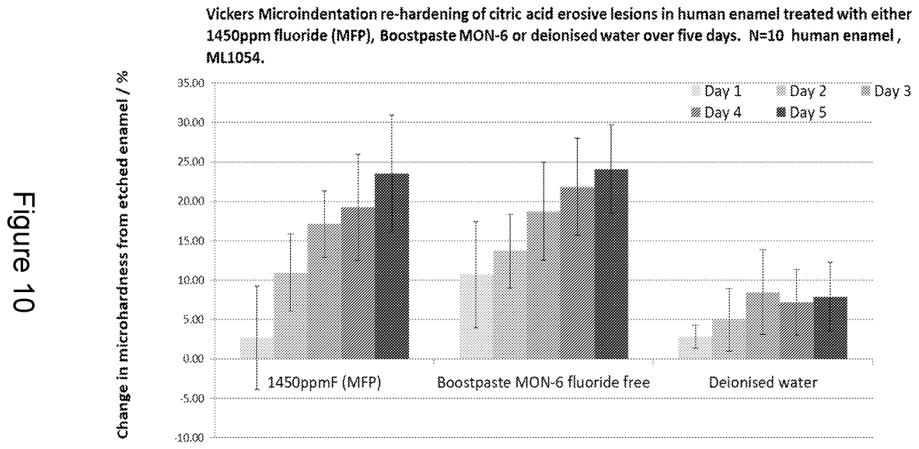

Results. The statistical analysis of the microindentation results shows that Boostpaste is statistically better at remineralizing erosive lesions compared to a 1450 ppm fluoride solution. After five days the calculated remineralisation from the Boostpaste treated enamel was over 66%. See FIG. 9.

### Example 11— Measurement of the Remineralisation of Citric Acid Erosive Lesions in Enamel after Treatment with Fluoride Free BoostPaste MON-06

Introduction. The remineralisation potential of three formulations: deionised water, 1450 ppm fluoride solution from MFP and fluoride free BoostPaste MON-6 formulation were tested in a standard microindentation erosive lesion rehardening model as described above in example 3. The objective of this study was to ascertain if fluoride free Boostpaste MON-6 formulation has the potential to remineralise erosive lesions without the presence of fluoride.

Method. Twice a day for five days human enamel specimens with citric acid erosive lesions were brushed with the test formulations and incubated in artificial saliva (containing 1500 units/L of phosphatase) at 37° C. between treatments. The microhardness of the enamel specimens was remeasured each day for five days and the degree of re-mineralization calculated using the boundary conditions of 100% mineralized for the hardness of the initial enamel before the formation of the lesions and 0% mineralization for the hardness of the enamel after the formation of the lesion.

Results. The statistical analysis of the microindentation results shows that fluoride free Boostpaste remineralizes erosive lesions, statistically equivalent to 1450 ppm fluoride solution. Both fluoride free BoostPaste and fluoride solution were statistically better than deionised water. After five days of treatment, the calculated remineralisation from the fluoride free BoostPaste treated enamel was over 24% and from the fluoride solution was over 23%. See FIG. 10.

### Example 12—Stability Study for MOL-05

Introduction: A laboratory scale stability study was performed on MOL-05 to test the stability of the formulation for total fluoride concentration, free fluoride concentration, free calcium concentration, the pH of the spray and light transmittance at 400 nm. A 500 ml batch of ToothBoost (MOL) was made and divided into two lots and decanted into spray packs. One lot was placed into a stability cabinet at 37.0±0.2° C. and 75.0±0.1% relative humidity. The second batch was stored at ambient temperature and humidity. Periodically, the samples were tested by selecting three spray packs at random from each of the two batches. The rationale for the tests chosen are: 1. Total fluoride to check the correct concentration of fluoride during manufacture. 2. Free fluoride to determine if fluoride and calcium are interacting in the formulation to form inactive calcium fluoride. 3. Free calcium to measure the dissociation of calcium from calcium-OPN complexes. 4. pH to monitor physical or chemical changes in the formulation. 5. Transmittance to monitor precipitation of insoluble material from the formulation.

Methods:
1. Total fluoride: Total fluoride was determined using method MT2222. An aliquot of ToothBoost was mixed with equal volume of 2M HCl and incubated overnight at 37° C. 2× volume of 1M NaOH was then added. The total volume was then doubled with TSAB (total ionic strength adjustment buffer). The pH was adjusted and the fluoride concentration measured using a fluoride ion selective electrode, calibrated against standard fluoride solutions prepared in the same way as above.
2. Free fluoride: Equal volumes of ToothBoost were mixed with TSAB and the fluoride concentration measured using a fluoride ion selective electrode, calibrated against standard fluoride solutions prepared in the same way.
3. Free calcium: Free calcium was determined using method MT2223. Equal volumes of ToothBoost were mixed with ISA (ion strength adjuster). The concentration of calcium was then measured using an ion selective electrode, calibrated against standard calcium solutions prepared in the same way as above.
4. pH: The pH of ToothBoost was measured directly using a sensitive pH electrode and normal conditions such as stir rate for the measurement.
5. Transmittance. A 1 cm square absorbance path quartz cuvette were filled directly with ToothBoost and absorbance measured at 400 nm wavelength. 400 nm had previously been identified as λ_{Max}.

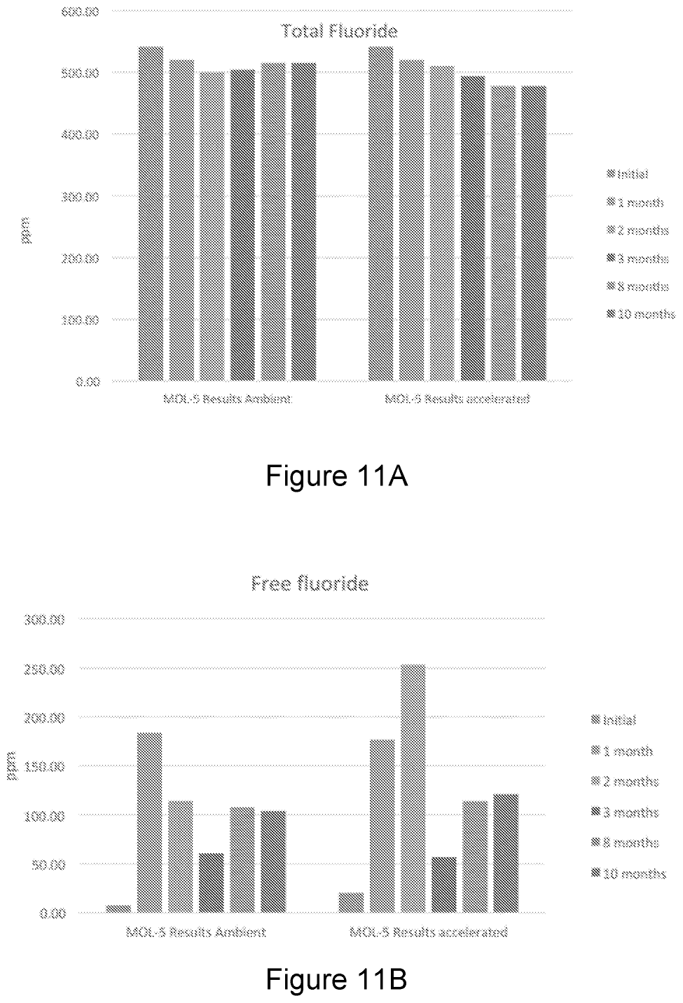

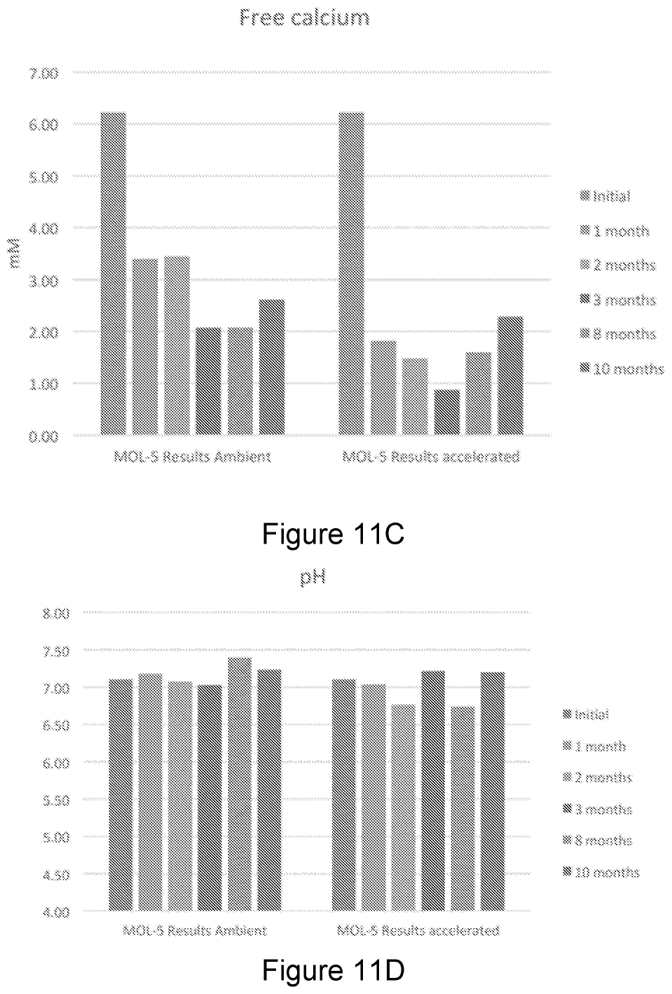

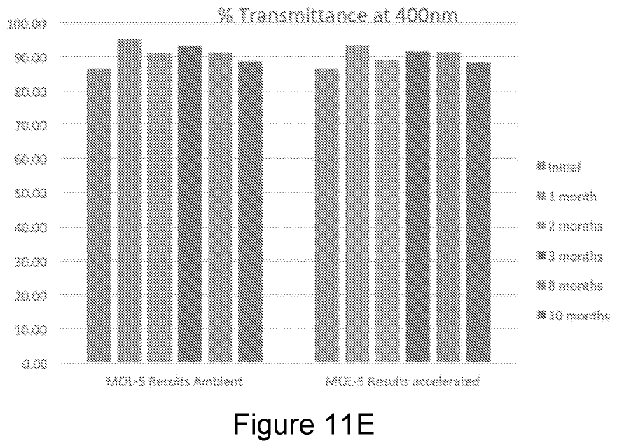

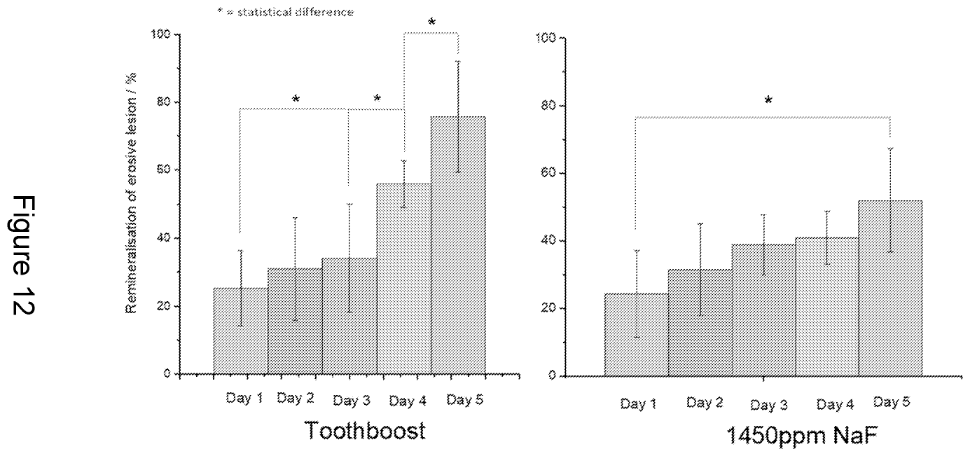

Results: The results are shown in FIGS. 11A-E. Total fluoride Reminded constant at ambient and accelerated conditions with an average of 514.28 SD15.2 ppm and 506.08 SD22.5 ppm respectively (FIG. 11A). Free fluoride starts at a minimum, quickly rises to a maximum and levels off to a constant value for the ambient and accelerated stored ToothBoost (FIG. 11B). Free calcium starts at a high concentration and gradually settles down to a constant value between 2.0 and 3.0 mM (FIG. 11C). The pH of the ambient and accelerated batches are constant over the 10 month stability observation period (FIG. 11D). % transmittance at 400 nm starts relatively low at around 87% (pure water has a transmittance of 100%). During the first month on stability the % T raises to around 90% ad maintains at that level for the remainder of the stability study (FIG. 11E).

Conclusion: After the manufacture of Tooth Boost, there is a period of organisation of the formulation where pH of ToothBoost may require some monitoring and adjusting. This lasts for 24 hours after manufacture. There is a longer period of up to one month where free fluoride, free calcium and the clarity of ToothBoost is in a state of change. The change then stabilises and ToothBoost remains in a state of good stability.

### Example 13— Study of Effect on Remineralisation of Erosive Lesions after Prolonged Use of Toothboost

N=6 human enamel specimens were challenged with a 1% citric acid solution for 10 minutes to form erosive lesions.
Every day for five days the lesions were treated with a single actuation of a spray pack with either a 1450 ppm fluoride solution (NaF) or ToothBoost (500 ppmF MFP).
After treatment the enamel was incubated in artificial saliva at 37° C.
The microhardness of the treated lesions were then measured after every day of treatment as described elsewhere herein

The degree of re-mineralization was calculated using the boundary conditions of 0% remineralisation of the lesions and 100% remineralisation for the initial sound enamel.

The results show that consistent use of a Toothboost oral spray causes a rapid and progressive remineralisation of erosive lesions compared to the industry standard sodium fluoride solution.

### Example 14— Study of Effect on Remineralisation of Subsurface Erosive Lesions after Use of Toothboost

Introduction: Quantitative Light Induced Fluorescence (QLF) was performed to measure the mineral density of subsurface lesions following treatment with Toothboost MOL-3 formulation. Artificial caries lesions were formed on human molars and which were then treated with Toothboost once a day for 50 days. The molars were maintained in artificial saliva throughout and periodically scanned using QLF to measure the mineral density lesion.

Method:
Subsurface lesions were formed on extracted human tooth specimens by preparing the specimens with a protective varnish, except for a diamond shaped window above the dentine/enamel junction for forming the caries lesion.
The specimens were placed into lactic acid and methyl cellulose gel for 10 days to form simulated subsurface lesions (International Dental Journal 2011; 61 (Suppl. 3): 55-59. David Churchley, Craig S. Newby, Richard Willson, Amir Haider, Bruce Schemehorn and Richard J. M. Lynch).
The molars were then treated with a single actuation of a spray pack of Toothboost MOL-3 formulation once a day for 50 days.
The specimens were incubated in artificial saliva at 37° C. in between treatment applications.
Once a week, the specimens were removed from the artificial saliva and qualitative light induced fluorescence (QLF) measurements were taken.
The QLF measurement indicates the density of the enamel within the diamond window.

Results: Toothboost progressively increased the mineral density over the treatment period until the caries lesion was remineralized to the density of the sound surrounding enamel at approximately day 40 (see FIG. 13a). The increase in mineral density of the caries lesion can be seen at 24 hr following application of Toothboost where the dark demineralised diamond window becomes lighter, with increased fluorescence, and is more similar to the surrounding sound enamel (see FIG. 13b).

1. A composition comprising a phosphopeptide for use in preventing or reducing staining of an oral surface.

2. A composition for use according to claim 1, wherein the composition is for use in combination with a whitening agent.

3. A composition comprising a phosphopeptide for use in prolonging the effect of a whitening agent on an oral surface.

4. A composition for use according to claim 2 or 3, wherein the oral surface is contacted with a whitening agent prior to contacting with the composition comprising a phosphopeptide.

5. A composition for use according to any of claims 1-4, wherein the composition does not comprise additional phosphate or an additional source of phosphate.

6. An oral care composition comprising a phosphopeptide, and optionally monofluorophosphate, wherein the composition does not comprise additional phosphate or an additional source of phosphate.

7. A composition comprising a phosphopeptide, and optionally monofluorophosphate, for use in remineralisation or prevention of demineralisation of an oral surface, wherein the composition does not comprise additional phosphate or an additional source of phosphate.

8. A composition comprising a phosphopeptide, and optionally monofluorophosphate, for use in the treatment or prevention of a dental disease or a dental condition involving dental demineralisation wherein the composition is for administration to the mouth of a subject, and wherein the composition does not comprise additional phosphate or an additional source of phosphate.

9. A composition comprising a phosphopeptide, and optionally monofluorophosphate, for use in remineralisation or prevention of demineralisation of an oral surface, or for the treatment or prevention of a dental disease or a dental condition involving dental demineralisation, by increasing the buffering capacity of saliva.

10. A composition for use according to any of claim 1-5, 7 8, or 9 wherein the composition is an oral care composition.

11. A composition for use according to any of claims 1-5, 7-10, or an oral care composition according to claim 6, wherein the phosphopeptide comprises osteopontin or phosphopeptides derived therefrom, and/or casein or phosphopeptides derived therefrom, preferably wherein phosphopeptide is OPN-10.

12. A composition for use according to any of claim 5, or 7-9, or an oral care composition according to claim 6, wherein the composition does not comprise additional phosphate or an additional source of phosphate other than the phosphopeptide and optionally monofluorophosphate, preferably wherein the composition does not comprise a phosphate buffer.

13. A composition for use according to any of claim 5, or 7-9, or an oral care composition according to claim 6, wherein the composition comprises less than 50 mM phosphate.

14. A composition for use according to any of claims 1-5, 7-13, or an oral care composition according to claim 6, wherein the composition further comprises fluoride or a source of fluoride, preferably the source of fluoride is monofluorophosphate.

15. A composition for use according to any of claims 1-5, 7-13, or an oral care composition according to claim 6, wherein the composition does not comprise fluoride or a source of fluoride.

16. A composition for use according to any of claims 1-5, 7-15, or an oral care composition according to claim 6, wherein the composition further comprises calcium or a source of calcium, preferably the source of calcium is calcium chloride.

17. A composition for use according to any of claims 1-5, 7-16, or an oral care composition according to claim 6, wherein the pH of the composition is between 7 and 9.

18. A composition for use according to any of claims 1-5, 7-17, or an oral care composition according to claim 6, wherein the composition comprises an aqueous medium.

19. A composition for use according to any of claims 1-5, 7-18, or an oral care composition according to claim 6, wherein the composition comprises about 20%, about 25%, about 30%, about 35%, about 40%, about 45%, about 50%, about 55%, about 60%, about 65%, about 70%, about 75%, about 80%, or about 90% by weight water.

20. A composition for use according to any of claims 1-5, 7-19, or an oral care composition according to claim 6, wherein the composition is a liquid and wherein the composition comprises at least 50% by weight water.

21. A composition for use according to any of claims 1-5, 7-19, or an oral care composition according to claim 6, wherein the composition is a colloid and wherein the composition comprises less than 25% by weight water.

22. A composition for use according to any of claims 1-5, 7-21, or an oral care composition according to claim 6, wherein the composition comprises one or more further components selected from: alcohol(s), humectant(s), surfactant(s), preservative(s), flavouring agent(s), sweetening agent(s), colouring agent(s), anti-caries agent(s), buffer(s), acid(s), base(s), whitening agent(s), thickener(s), and anticalculus agent(s).

23. A composition for use according to any of claims 1-5, 7-22, or an oral care composition according to claim 6, wherein the composition comprises any of the following components selected from:
Water from about 20% to about 99% by weight; preferably from about 23% to about 66% by weight;
A buffer from about 1% to about 20% by weight; preferably from about 1% to about 15% by weight;
Optionally a source of calcium ions from about 0.1% to about 15% by weight; preferably from about 0.1% to about 5% by weight;
Optionally a source of phosphate ions from about 0.2% to about 15% by weight;
preferably from about 0.5% to about 5% w/v, preferably from about 0.7% to about 2% by weight;
A phosphopeptide from about 0.5% to about 15% by weight; suitably from about 1% to about 10% by weight, preferably from about 1.5% to about 5% by weight;
Optionally a source of fluoride from about 0.01% to about 3% by weight; preferably from about 0.1% to about 1.5% by weight, preferably from about 0.4% to about 1.5% by weight;
A flavouring, preservative and/or other ingredients from about 0% to about 70% by weight; preferably from about 0% to about 20% by weight; preferably from about 0% to about 10% by weight;
A sweetener from about 0.1% to about 20% by weight; preferably from about 0.1% to about 10% by weight;
Optionally an acid from about 5-40% by weight; preferably from about 10-35% by weight;
Optionally a whitening agent from about 1-20% by weight; suitably from about 5-10% by weight; and
Optionally a thickener from about 0.1-20% by weight, suitably from about 0.5-15% by weight.

24. A composition for use according to any of claims 1-5, 7-22, or an oral care composition according to claim 6, wherein the composition comprises the following components:
A phosphopeptide in an amount of about 3% w/w, preferably wherein the phosphopeptide is OPN-10
A buffer in an amount of about 15% w/w, preferably wherein the buffer is sodium bicarbonate
Water in an amount of about 54 to 66% w/w, preferably wherein the water is deionised water
A source of calcium in an amount of about 3% w/w, preferably wherein the source of calcium is a calcium chloride solution
Optionally a source of fluoride in an amount of about 0.4% w/w, preferably wherein the source of fluoride is monofluorophosphate
A sweetener in an amount of about 5% w/w, preferably wherein the sweetener is xylitol
Optionally an acid in an amount of about 11% w/w, preferably wherein the acid is hydrochloric acid
A flavouring and preservative agent in an amount of about 8% w/w, preferably wherein the agent comprises a mixture of sodium methyl paraben, phenoxyethanol, saccharine, tego betain, and flavour oil.

25. A composition for use according to any of claims 1-5, 7-22, or an oral care composition according to claim 6, wherein the composition comprises the following components:
A phosphopeptide in an amount of about 3% w/w; preferably wherein the phosphopeptide is OPN-10
A buffer in an amount of about 2% w/w, preferably wherein the buffer is sodium bicarbonate
A whitening agent in an amount of about 6% w/w, preferably wherein the whitening agent comprises abrasive silica
Water in an amount of about 23 to 25% w/w, preferably wherein the water is deionised water
An acid in an amount of about 34% w/w, preferably wherein the acid is hydrochloric acid
A source of calcium in an amount of about 0.5% w/w, preferably wherein the source of calcium is calcium chloride
Optionally a source of fluoride in an amount of about 1% w/w, preferably wherein the source of fluoride is monofluorophosphate
A sweetener in an amount of about 9% w/w, preferably wherein the sweetener is mannitol
A thickener in an amount of about 13% w/w, preferably wherein the thickener is xanthan gum and silica
A flavouring and preservative agent in an amount of about 8% w/w, preferably wherein the agent comprises a mixture of sodium methyl paraben, phenoxyethanol, saccharine, tego betain, and flavour oil.

26. A composition for use according to claim 1, 3 or 7, wherein the oral surface is a soft or hard surface, and/or wherein the oral surface is a natural or synthetic surface.

27. A composition for use according to claim 7 or 9 wherein the oral surface is a mineralized surface.

28. A composition for use according to claim 1, 3, 7, or 9 wherein the oral surface is the enamel of a tooth.

29. A composition for use according to claim 1, 3, 7, 8 or 9 wherein the composition is for administration to the subject at least once per day, preferably at least twice or three times per day, and/or wherein the composition is for administration to the mouth of the subject.

30. A composition for use according to claim 1, 3, 7, 8, 9 or 29 wherein the composition is for administration to a mouth of the subject, at least twice per day for at least 5 days.

31. Packaging comprising an oral care composition according to claim 6, or an oral care composition for use according to any of claims 1-5, 7-30.

32. Packaging according to claim 31, wherein the packaging comprises an actuator operable to deliver a metered dose of the oral care composition when actuated.

33. Packaging according to claim 32, wherein the actuator comprises a spray nozzle.
